(19) United States
(12) Patent Application Publication
(10) Pub. No.: US 2008/0213322 A1
(43) Pub. Date: Sep. 4, 2008

(54) Method for the hydrophobic treatment of pigment particles and the personal care compositions comprising said pigment particles

(76) Inventors: Michael Birman, West Orange, NJ (US); Oliver Reich, Grenzach-Wyhlen (DE); Joseph A. Lupia, Monroe, NY (US); Jerry L. Pool, Greensboro, NC (US)

Correspondence Address: JoAnn Villamizar;Ciba Corporation/Patent Department, 540 White Plains Road, P.O. Box 2005, Tarrytown, NY, 10591, US

(21) Appl. No.: 11/903,595

(22) Filed: Sep. 24, 2007

Related U.S. Application Data
(60) Provisional application No. 60/849,563, filed on Oct. 5, 2006.
(60) Provisional application No. 60/854,306, filed on Oct. 25, 2006.

(51) Int. Cl. A61K 8/02; C04B 16/12; C04B 14/20; A61Q 3/00; A61Q 17/04; C04B 16/04; A61K 8/89

(52) U.S. Cl. 424/401; 106/481; 523/200; 106/417; 106/475; 514/63; 424/59; 424/61; 424/78.08

(57) ABSTRACT

The present invention provides a method for the hydrophobic treatment of pigment particles comprising contacting said pigment particles with one or more silicone-based materials, that are substantially free of silicon-hydrogen bonds and fluorine, producing hydrophobic, non-covalently bound, modified pigment particles. Additionally, the present invention provides personal care compositions comprising said modified or coated pigment particles.

This application claims benefit under 35 USC 119(e) of U.S. Provisional app. No. 60/849,563, filed on Oct. 5, 2006 and U.S. Provisional app. No. 60/854,306, filed on Oct. 25, 2006, both of which are incorporated herein by reference.

### FIELD OF THE INVENTION

This invention relates to a method of treating pigment particles to increase their hydrophobicity and to personal care compositions comprising said modified or coated pigment particles.

### BACKGROUND OF THE INVENTION

Insoluble pigment materials or particles, for example colorful pigments, sunscreen agents, talc and the like, are commonly employed in the cosmetics and other industries, such as paint, coatings, and plastics industries, to serve a variety of purposes. Suitable pigments or pigment particles may impart qualities of color, opacity or special visual effects, such as pearlescence, or other qualities such as bulk, free and oil absorbency, to a wide range of consumer and industrial products. Such pigments are generally insoluble in either aqueous or organic media. The invention will be described herein as it applies to personal care products, with the understanding that the novel materials, methods, and compositions of matter provided by the invention may be useful in other industries where such modified pigments may be employed, as will be apparent to those skilled in the art from this disclosure.

Pigment particles of interest to the invention are well divided particles which are intended to be uniformly dispersed in the finished product. Defined particle size and uniformity of dispersion are desirable characteristics that contribute to the quality of the finished product and to efficient utilization of the pigment. Finer pigment particles expose more surface area of particle material in the end product, enabling the particles' color or other property to be more efficiently imparted to the finished product. Larger particles exhibit better coverage power or, in case of effect pigments, greater luster and sparkle. Uniform dispersion of the particles in liquid or even pigment excipients is desirable or even essential to provide a consistent commercial product with good shelf life which is free of discoloration, settling or other blemishes.

A number of difficulties may arise in uniformly dispersing pigment particles, especially finely divided pigments. Untreated, many pigments, for example metal oxides such as iron oxide, titanium dioxide and zinc oxide, have significant surface reactivity which may be attributable to chemical reactivity either covalent or ionic, or to more physical phenomena such as adsorbability or accumulation of surface charge. Such surface reactivity may interfere with the uniformity of an initial dispersion of the powder or may adversely impact the long-term stability of the end product. The pigment particles may tend to couple covalently or electrochemically with other ingredients in the formulation or to agglomerate, which is to say to stick to each other in agglomerations or clumps. The result may be a poor or unacceptable end product or a product which has limited shelf life owing to non-uniformity of color or other properties, agglomeration, a poor, gritty or sandy feel, settling and so on.

Additionally surface tension and the hydrophilic nature of untreated inorganic pigments complicate incorporation into lipophilic systems, while strong oil absorption results in a negative impact on viscosity.

Finally untreated pigment particles, especially light particles of low density, cause serious dusting problems during handling of bulk material.

To overcome these problems, it has long been customary to surface treat pigments to render them hydrophobic and to enhance the handling, processing and performance of the pigment particles in finished products. Typical coatings work by reducing the surface activity of the pigment particles, repelling water or other aqueous media, inhibiting agglomeration, reducing oil absorption and enhancing dispersibility of the powder particles in aqueous or oily media used in formulating finished products. A satisfactory coating should cover each particle completely and more or less uniformly.

To these ends, many hydrophobic coatings and treatments are commercially available and have been proposed in the literature, especially in the patent literature. Many may be effective for some purposes on one or a small number of pigments, but no treatment known to the applicants is effective on a full range of pigment particles.

Insoluble pigment particles include many quite different materials such as metal oxides, metal silicates, other inorganic salts, pigment extenders or fillers such as talc and silica as well as organic materials such as lakes, which are organic dyes fixed on metallic salts or oxides, and other materials, as is well known in the art. These materials have a variety of surface properties, and a single personal care composition or formulation may use a number of different such pigment or other cosmetic powder ingredients, having a number of different coatings. However, the different coatings may interact undesirably with one another. Therefore, to avoid interactions and for simplicity, it would be desirable for all the particulates in a given formula to receive the same treatment. It would be still more desirable to have a single hydrophobic treatment which was effective for most regularly used pigments.

Additionally, specific inorganic pigments lack affinity to skin. Hydrophobic modification enhances the skin affinity and adhesion of the pigment to the skin, resulting in a better coloring result of the cosmetic formulations on the skin, and in better wearing properties; for example, longer lasting effects. Also, the overall skin feel is improved. However, especially big particles, like high sparkling effect pigments, can still feel raspy on the skin even with state-of-the-art hydrophobic modifications. It would thus be beneficial if a treatment existed that is capable of providing a smooth, silky skin feel to big particles—without causing a waxy or tacky sensation.

Witucki in “A Silane Primer: Chemistry and Applications of Alkoxy Silanes” Journal of Coatings Technology 65; 822 pages 57-60 (July 1993) discusses use of alkoxy functional silanes for surface treatment of inter alia particulate pigments and fillers. Described reaction mechanisms include hydrogen bonding to surface hydroxy groups followed by drying or curing with elimination of water to form a covalent bond from each alkoxy-bearing silicon atom to the particle substrate.

U.S. Pat. No. 4,801,445 is directed to a modified powder or particulate material having a silicone polymer film coated onto the surface herein incorporated by reference.

U.S. Pat. No. 3,649,321 is directed to the coating of titanium dioxide particles with a silicone and at least one ester herein incorporated by reference.

U.S. Pat. No. 3,132,961 is directed to coating fillers, pigments and the like with diorganopolysiloxanes herein incorporated by reference.

US 2004-0096470 claims a cosmetic composition comprising, among other things, a double coated pigment which is coated by a mixture of a fluorine coating compound and a silicone coating compound herein incorporated by reference.

US 2004-0197286 discloses a compacted cosmetic powder composition comprising: 1) at least one polyester comprising at least one fatty acid ester of fatty alcohol ester, comprising at least two saturated and branched carbonaceous chains of 24 to 28 carbon atoms, 2) at least one silicone compound, and 3) a particulate phase comprising at least one pulverulent compound, wherein the compacted cosmetic powder composition is homogenous. The application is herein incorporated by reference.

U.S. Pat. No. 5,738,841 discloses a care product and/or a makeup product for the skin and/or keratinous material comprising an anhydrous composition comprising, in a fatty phase, at least one silicone containing compound and octyldodecyl neopentanoate, wherein the product is applied to the skin and or keratinous material in the anhydrous form. The application is herein incorporated by reference.

JP 2000-212024, JP 2000-256133, JP 2001-302455, JP 2001-316223, JP 1998-134442, JP 1996-118051, JP 1991-220446, and JP 1990-63913 disclose cosmetic compositions comprising cosmetic pigments treated with perfluorinated silicone derivatives and/or perfluorinated phosphate ester derivatives.

U.S. Pat. No. 6,417,250 discloses a formulation comprising a film-former or casting resin, characterized in that it contains pearl luster pigments which are coated with one or more silanes herein incorporated by reference.

US 2004-0234613 discloses hybrid coating material and process for pigments and other powders, for example cosmetic powders employ an organometallate, for example a titanante, and a functionalized silicon compound, which is covalently bond to each other and to the substrate powder herein incorporated by reference.

US 2003-0161805 discloses a cosmetic powder treatment with a linear reactive alkylpolysiloxane having substituted in repeating units in the backbone of the molecule both cationic and anionic groups, for example aminoethylaminopropyl and alkoxy groups herein incorporated by reference.

US 2004-0223929 discloses personal care compositions comprising a hydrophobically modified interference pigment herein incorporated by reference.

U.S. Pat. No. 5,143,722 describes the coating of cosmetic pigments with hydrophobic materials comprising dimethylpolysiloxane materials, including cross-linked products herein incorporated by reference.

JP7-196946 discloses the use of a straight chain alkylpolysiloxanes having reactive terminal groups such as alkoxy, hydroxy, halogen, amino or imino groups for treating pigments.

U.S. Pat. No. 5,458,681 discloses a similar reactive approach where alkylpolysiloxanes with a specific narrow distribution of molecular weight are employed, namely a ratio of weight-average molecular weight to number average molecular weight of from 1.0 to 1.3 herein incorporated by reference.

U.S. Pat. No. 6,524,705, JP55-136213, and JP56-016404 disclose pigment particles treated with a silanic hydrogen containing silicone derivative, the US reference herein incorporated by reference.

Use of organometallic catalysts to catalyze a surface coating reaction is undesirable, because the presence of materials containing extraneous heavy metals in cosmetic products that are applied to the human body is unacceptable.

Methyl hydrogen polysiloxane has Si—H groups which can react with hydroxy groups on the pigment surface. During the coating process, methyl hydrogen polysiloxane may undergo polymerization to form a cross linked resin coating the particles and possibly also causing cohesive aggregation of the pigments. In this process, the Si—H groups in the methyl hydrogen polysiloxane cannot completely react owing to conformational energy barriers. Residual Si—H groups may then react with the pigment gradually over time, or with other ingredients in the finished product, to release hydrogen, spoiling the integrity of the product.

U.S. Pat. No. 6,200,580 discloses, inter alia use of a reactive alkyl polysiloxane having a single, terminal reactive group, which can be an amino group to coat powdered base materials including sericite herein incorporated by reference.

A further drawback to the use of methyl hydrogen polysiloxanes, such as Dow Corning (trademark) product #1107, as coating materials for cosmetic powders, is the limited range of materials they can coat. For example, methyl hydrogen polysiloxane does not bond well to lakes of organic colorant such as D&C Red No. 6 Barium Lake and the resulting water repellency is poor. Thus, methyl hydrogen polysiloxanes are unsatisfactory coating materials for cosmetic powders.

Another class of materials that are hard to coat is mica-based materials such as sericites which are favored in cosmetics for their pearlescence. Even an alkoxysilane, such as SILQUEST (trademark) A-137 silane from OSI Specialties, Greenwich Conn., one of the more reactive silicone starting materials, does not react well with sericite and the resultant hydrophobicity is not satisfactory.

U.S. Pat. No. 6,176,918 discloses a method of coating mica-based modified pearl luster pigments employing an oligomeric silane system herein incorporated by reference.

Accordingly, there is still a need for a hydrophobic treatment process for coating a wide range of cosmetic powder materials which process, preferably can also be used to effectively treat hard-to-coat pigment particles. There is a further need for such modified pigment particles in personal care compositions and other formulations.

### SUMMARY OF THE INVENTION

One object of the present invention provides a method for the hydrophobic treatment of pigment particles comprising contacting said pigment particles with one or more silicone-based materials that are substantially free of silicon-hydrogen bonds and fluorine, producing hydrophobically modified pigment particles. Another object of the present invention is personal care composition comprising said modified pigment particles.

### DETAILED DESCRIPTION OF THE INVENTION

As stated, it is the one aspect of this invention to provide a method for the hydrophobic treatment of pigment particles comprising contacting said pigment particles with an effective amount of one or more silicone compounds having formula (1), (2), and (3):

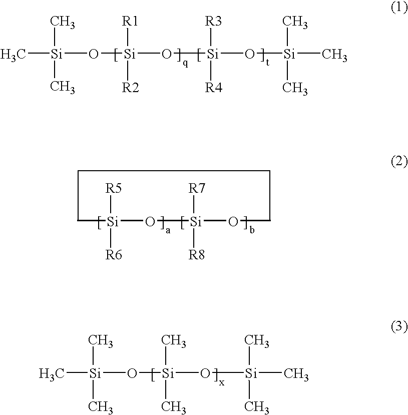

wherein R1 is C1 to C22 branched or straight chain alkyl or aryl of six to ten carbon atoms; R2 is C1 to C22 branched or straight chain alkyl or aryl of six to ten carbon atoms; R3 is C1 to C22 branched or straight chain alkyl or aryl of six to ten carbon atoms; R4 is C11 to C22 branched or straight chain alkyl or aryl of six to ten carbon atoms; q is from 1 to about 2000; t is from 1 to about 2000; the sum of q+t is from 2 to about 2000;
R5 is C1 to C22 branched or straight chain alkyl or aryl of six to ten carbon atoms; R6 is C1 to C22 branched or straight chain alkyl or aryl of six to ten carbon atoms; R7 is C1 to C22 branched or straight chain alkyl or aryl of six to ten carbon atoms; R8 is C1 to C22 branched or straight chain alkyl or aryl of six to ten carbon atoms; a is from 1 to about 100; b is from 1 to about 100; the sum of a+b is from 2 to about 100;
x is an integer from 1 to about 2000;
with the proviso that formula (1), (2), and (3) are essentially free of silicon-hydrogen bonds and fluorine and with the further proviso that one of the silicone compounds is of formula (1).

Another embodiment of the instant invention is a method for the hydrophobic treatment of pigment particles comprising contacting said pigment particle with an effective amount of one or more silicone compounds having formula (1), (2), and (3) wherein the silicone compound of formula (1) is defined as:

R1 is C1 to C10 branched or straight chain alkyl or aryl of six to ten carbon atoms; R2 is C1 to C10 branched or straight chain alkyl or aryl of six to ten carbon atoms; R3 is C1 to C10 branched or straight chain alkyl or aryl of six to ten carbon atoms; R4 is C12 to C18 branched or straight chain alkyl or aryl of six to ten carbon atoms; q is from 1 to about 1000; t is from 1 to about 1000; the sum of q+t is from 2 to about 1000.

Another embodiment of the instant invention is a method for the hydrophobic treatment of pigment particles comprising contacting said pigment particle with an effective amount of one or more silicone compounds having formula (1), (2), and (3) wherein the silicone compound of formula (1) is defined as:

R1 is C1 to C6 branched or straight chain alkyl; R2 is C1 to C6 branched or straight chain alkyl; R3 is C1 to C16 branched or straight chain alkyl; R4 is C12 to C18 branched or straight chain alkyl; q is from 1 to about 100; t is from 1 to about 100; the sum of q+t is from 2 to about 100.

Another embodiment of the instant invention is a method for the hydrophobic treatment of pigment particles comprising contacting said pigment particle with an effective amount of one or more silicone compounds having formula (1), (2), and (3) wherein the silicone compound of formula (1) is cetyldimethicone.

Another embodiment of the instant invention is a method for the hydrophobic treatment of pigment particles comprising contacting said pigment particle with an effective amount of one or more silicone compounds having formula (1), (2), and (3) wherein the silicone compound of formula (2) is defined as:

R5 is C1 to C6 branched or straight chain alkyl; R6 is C1 to C6 branched or straight chain alkyl; R7 is C1 to C6 branched or straight chain alkyl; R8 is C1 to C16 branched or straight chain alkyl; a is from 1 to about 25; b is from 1 to about 25; the sum of a+b is from 2 to about 25.

Another embodiment of the instant invention is a method for the hydrophobic treatment of pigment particles comprising contacting said pigment particle with an effective amount of one or more silicone compounds having formula (1), (2), and (3) wherein the silicone compound of formula (2) is defined as:

R5 is methyl; R6 is methyl; R7 is methyl; R8 is C1 to C8 straight chain alkyl; a is from 1 to about 9; b is from 1 to about 9; the sum of a+b is from 3 to about 9.

Another embodiment of the instant invention is a method for the hydrophobic treatment of pigment particles comprising contacting said pigment particle with an effective amount of one or more silicone compounds having formula (1), (2), and (3) wherein the silicone compound of formula (2) is cyclotetradimethylsiloxane, cyclopentadimethylsiloxane, cyclohexadimethylsiloxane, cyclocopolymer of dimethylsiloxane/methyloctylsiloxane, hexylheptamethyltrisiloxane or octylheptamethyltrisiloxane.

Another embodiment of the instant invention is a method for the hydrophobic treatment of pigment particles comprising contacting said pigment particle with an effective amount of one or more silicone compounds having formula (1), (2), and (3) wherein the silicone compound of formula (3) is defined as:

x is an integer from 1 to about 500.

Another embodiment of the instant invention is a method for the hydrophobic treatment of pigment particles comprising contacting said pigment particle with an effective amount of one or more silicone compounds having formula (1), (2), and (3) wherein the silicone compound of formula (3) is defined as:

x is an integer from 1 to about 50.

Another embodiment of the instant invention is a method for the hydrophobic treatment of pigment particles comprising contacting said pigment particle with an effective amount of one or more silicone compounds having formula (1), (2), and (3) wherein the silicone compound of formula (3) is dimethylpolysiloxane.

Another embodiment of the instant invention is a method for the hydrophobic treatment of pigment particles comprising contacting said pigment particle with an effective amount of cetyldimethicone and silicone compounds having formula (2) and/or (3) wherein the silicone compound of formula (2) is defined as:

R5 is C1 to C6 branched or straight chain alkyl; R6 is C1 to C6 branched or straight chain alkyl; R7 is C1 to C6 branched or straight chain alkyl; R8 is C1 to C16 branched or straight chain alkyl; a is from 1 to about 25; b is from 1 to about 25; the sum of a+b is from 2 to about 25.

Another embodiment of the instant invention is a method for the hydrophobic treatment of pigment particles comprising contacting said pigment particle with an effective amount of cetyldimethicone and silicone compounds having formula (2) and/or (3) wherein the silicone compound of formula (2) is defined as:

R5 is methyl; R6 is methyl; R7 is methyl; R8 is C1 to C8 straight chain alkyl; a is from 1 to about 9; b is from 1 to about 9; the sum of a+b is from 3 to about 9.

Another embodiment of the instant invention is a method for the hydrophobic treatment of pigment particles comprising contacting said pigment particle with an effective amount of cetyldimethicone and silicone compounds having formula (2) and/or (3) wherein the silicone compound of formula (2) is, for example, cyclotetradimethylsiloxane, cyclopentadimethylsiloxane, cyclohexadimethylsiloxane, cyclocopolymer of dimethylsiloxane/methyloctylsiloxane, hexylheptamethyltrisiloxane or octylheptamethyltrisiloxane.

Another embodiment of the instant invention is a method for the hydrophobic treatment of pigment particles comprising contacting said pigment particle with an effective amount of cetyldimethicone and silicone compounds having formula (2) and/or (3) wherein the silicone compound of formula (3) is defined as:

x is an integer from 1 to about 500.

Another embodiment of the instant invention is a method for the hydrophobic treatment of pigment particles comprising contacting said pigment particle with an effective amount of cetyldimethicone and silicone compounds having formula (2) and/or (3) wherein the silicone compound of formula (3) is defined as:

x is an integer from 1 to about 50.

Another embodiment of the instant invention is a method for the hydrophobic treatment of pigment particles comprising contacting said pigment particle with an effective amount of cetyldimethicone and silicone compounds having formula (2) and/or (3) wherein the silicone compound of formula (3) is dimethylpolysiloxane.

The silicones used in the present invention are, in general, commercially available polysiloxanes, for example, alkyl or aryl substituted polysiloxanes. Examples of silicones according to formula (1) include cetyldimethicone. Examples of silicones according to formula (2) include cyclotetradimethylsiloxane, cyclopentadimethylsiloxane, cyclohexadimethylsiloxane, cyclocopolymer of dimethylsiloxane/methyloctylsiloxane, hexylheptamethyltrisiloxane and octylheptamethyltrisiloxane. Examples of silicones according to formula (3) include dimethylpolysiloxane. A mixture of silicones of formula (1), (2), and/or (3) may be used according to the method of the instant invention; for example, a mixture of any two or more of silicones according to formula (1), (2), and/or (3).

Another embodiment of the instant invention is a method for the hydrophobic treatment of pigment particles wherein the weight ratio of formula (1) to formula (2) and/or formula 3 is from about 1000:1 to about 1:1000.

Another embodiment of the instant invention is a method for the hydrophobic treatment of pigment particles wherein the weight ratio of formula (1) to formula (2) and/or formula 3 is from about 100:1 to about 1:100.

Another embodiment of the instant invention is a method for the hydrophobic treatment of pigment particles wherein the weight ratio of formula (1) to formula (2) and/or formula 3 is from about 10:1 to about 1:10.

The term “essentially free of” refers to values less than about 1 percent based on weight; preferably to values less than about 0.1 percent based on weight; more preferably to values less than about 0.01 percent by weight; and, most preferably refers to zero percent based on weight.

The silicone should be used in the liquid state. Normally solid or waxy silicones may be employed in a solution with a suitable solvent, for example, acetone or ethanol; or may be heated above their corresponding melting or softening temperature and pigment treatment carried above these temperatures. The silicone should have sufficiently low vapor pressure that it is not substantially removed from the pigment by the drying process, for example at 12° C. and, as plastics compositions incorporating the modified pigment may be used in injection molding processes which operate at temperatures of about 200 C or higher, the silicone should have a sufficiently low vapor pressure and also be stable at these temperatures.

An effective amount of the silicone compounds of formula (1), (2), and (3) refers to the amount of silicone compounds required to effectively treat the untreated pigment particle surface. An effective amount of the silicone compounds according to the instant method is in the range from about 0.1 percent to about 90 percent based on the weight of the modified or treated pigment particle. An effective amount of the silicone compounds according to the instant method is in the range from about 0.1 percent to about 30 percent based on the weight of the modified or treated pigment particle. An effective amount of the silicone compounds according to the instant method is in the range from about 1 percent to about 15 percent based on the weight of the modified or treated pigment particle. An effective amount of the silicone compounds according to the instant method is in the range from about 3 percent to about 10 percent based on the weight of the modified or treated pigment particle.

Although there are no critical limitations to the sizes of the pigment particles, preferably pigment particles having an average size from about 0.01 to about 500 micrometers, more preferably having an average size of about 1 to about 200 micrometers, most preferably having an average size from about 5 to about 150 micrometers are modified with the silicone compounds according to formula (1), (2), or (3) of the instant invention.

Although there are no critical temperature limitations, the method according to the instant invention is carried out at temperatures from about 0 C to about 200 C; preferably from about 10 C to about 150 C; and more preferably from about 25 C to about 125 C.

According to the method of the instant invention, the silicone compounds of formula (1), (2) and/or (3) may be incorporated on the untreated pigment particle surface by a number of different ways. Spray drying and jet milling are two ways to introduce the silicone compounds of formula (1), (2), and/or (3) onto the untreated pigment particle surface.

Additionally, the silicone compounds of formula (1), (2), and/or (3) may be dissolved in an appropriate solvent and then followed by adequate slurrying the untreated pigment particles into the solution. The resulting modified pigment particles are removed by filtration or centrifugation followed by drying to remove residual solvent.

It will often be desirable to coat the pigment particles with, for example, one or more hydrous inorganic oxides, for example, hydrated silica or alumina, TiO2, Fe-oxides, tin oxide, mica, zinc oxide, colorants lake carmine or Prussian blue. In this case the treatment by the method of the instant invention will be carried out after the particles have been coated with, for example, the hydrous inorganic oxide.

In another embodiment of the instant invention, the pigment particles are coated with one or more hydrous inorganic oxides, classified and washed to remove any soluble substances and are then treated with the silicone, the modified pigment being subsequently dried and milled. Alternatively, the pigment particle coated with the hydrous inorganic oxides may be dried and/or calcined partially to dehydrate the hydrous inorganic oxides and, if desired, milled before treatment with the silicone, either in solution or in the vapor phase.

Another embodiment of the instant invention is a method for the hydrophobic treatment of pigment particles comprising contacting said pigment particles with an effective amount of one or more silicone compounds having formula (1), (2), and/or (3) wherein the pigment particles are selected from the group consisting of inorganic pigments, metal oxides and hydroxides, mica, organic pigments, pearlescent pigments, mineral silicates, porous materials, carbons, and interference pigments.

Another embodiment of the instant invention is a method for the hydrophobic treatment of pigment particles comprising contacting said pigment particles with an effective amount of one or more silicone compounds having formula (1), (2), and/or (3) wherein the pigment particles are selected from the group consisting of inorganic pigments, metal oxides and hydroxides, mica, organic pigments, pearlescent pigments, and interference pigments.

Another embodiment of the instant invention is a method for the hydrophobic treatment of pigment particles comprising contacting said pigment particles with an effective amount of one or more silicone compounds having formula (1), (2), and/or (3) wherein the pigment particles are selected from the group consisting of inorganic pigments, organic pigments, pearlescent pigments, and interference pigments.

Another embodiment of the instant invention is a method for the hydrophobic treatment of pigment particles comprising contacting said pigment particles with an effective amount of one or more silicone compounds having formula (1), (2), and/or (3) wherein the pigment particles are pearlescent pigments or interference pigments.

According to the instant invention, various kinds of pigment particles can be modified by coating the surfaces thereof with a silicone compound. Examples of pigment particles that can be treated according to the instant invention, but is not limited to, include inorganic pigments, metal oxides and hydroxides, mica, organic pigments, pearlescent pigments, mineral silicates, porous materials, carbons, interference pigments, and the like.

Another embodiment of the instant invention is a coated pigment particle comprising a pigment particle and the coating on said pigment particle wherein said coating comprises and effective amount of one or more silicone compounds having formula (1), (2), and (3):

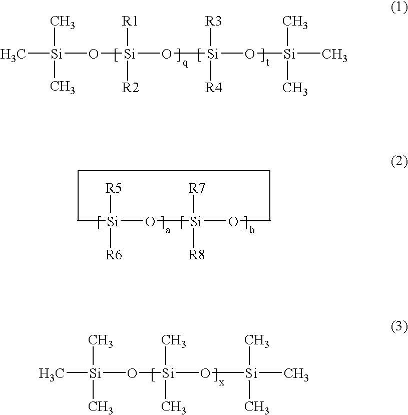

wherein R1 is C1 to C22 branched or straight chain alkyl or aryl of six to ten carbon atoms; R2 is C1 to C22 branched or straight chain alkyl or aryl of six to ten carbon atoms; R3 is C1 to C22 branched or straight chain alkyl or aryl of six to ten carbon atoms; R4 is C11 to C22 branched or straight chain alkyl or aryl of six to ten carbon atoms; q is from 1 to about 2000; t is from 1 to about 2000; the sum of q+t is from 2 to about 2000;
R5 is C1 to C22 branched or straight chain alkyl or aryl of six to ten carbon atoms; R6 is C1 to C22 branched or straight chain alkyl or aryl of six to ten carbon atoms; R7 is C1 to C22 branched or straight chain alkyl or aryl of six to ten carbon atoms; R8 is C1 to C22 branched or straight chain alkyl or aryl of six to ten carbon atoms; a is from 1 to about 100; b is from 1 to about 100; the sum of a+b is from 2 to about 100;
x is an integer from 1 to about 2000;
with the proviso that formula (1), (2), and (3) are essentially free of silicon-hydrogen bonds and fluorine and with the further proviso that at least one of the silicone compounds is of formula (1).

Another embodiment of the instant invention is a coated pigment particle comprising a pigment particle and the coating on said pigment particle wherein said coating comprises one or more silicone compounds having formula (1), (2), and (3) wherein the silicone compound of formula (1) is defined as:

R1 is C1 to C10 branched or straight chain alkyl or aryl of six to ten carbon atoms; R2 is C1 to C10 branched or straight chain alkyl or aryl of six to ten carbon atoms; R3 is C1 to C10 branched or straight chain alkyl or aryl of six to ten carbon atoms; R4 is C12 to C18 branched or straight chain alkyl or aryl of six to ten carbon atoms; q is from 1 to about 1000; t is from 1 to about 1000; the sum of q+t is from 2 to about 1000.

Another embodiment of the instant invention is a coated pigment particle comprising a pigment particle and the coating on said pigment particle wherein said coating comprises one or more silicone compounds having formula (1), (2), and (3) wherein the silicone compound of formula (1) is defined as:

R1 is C1 to C6 branched or straight chain alkyl; R2 is C1 to C6 branched or straight chain alkyl; R3 is C1 to C16 branched or straight chain alkyl; R4 is C12 to C18 branched or straight chain alkyl; q is from 1 to about 100; t is from 1 to about 100; the sum of q+t is from 2 to about 100.

Another embodiment of the instant invention is a coated pigment particle comprising a pigment particle and the coating on said pigment particle wherein said coating comprises one or more silicone compounds having formula (1), (2), and (3) wherein the silicone compound of formula (1) is cetyldimethicone.

Another embodiment of the instant invention is a coated pigment particle comprising a pigment particle and the coating on said pigment particle wherein said coating comprises one or more silicone compounds having formula (1), (2), and (3) wherein the silicone compound of formula (2) is defined as:

R5 is C1 to C6 branched or straight chain alkyl; R6 is C1 to C6 branched or straight chain alkyl; R7 is C1 to C6 branched or straight chain alkyl; R8 is C1 to C16 branched or straight chain alkyl; a is from 1 to about 25; b is from 1 to about 25; the sum of a+b is from 2 to about 25.

Another embodiment of the instant invention is a coated pigment particle comprising a pigment particle and the coating on said pigment particle wherein said coating comprises one or more silicone compounds having formula (1), (2), and (3) wherein the silicone compound of formula (2) is defined as:

R5 is methyl; R6 is methyl; R7 is methyl; R8 is C1 to C8 straight chain alkyl; a is from 1 to about 9; b is from 1 to about 9; the sum of a+b is from 3 to about 9.

Another embodiment of the instant invention is a coated pigment particle comprising a pigment particle and the coating on said pigment particle wherein said coating comprises one or more silicone compounds having formula (1), (2), and (3) wherein the silicone compound of formula (2) is cyclotetradimethylsiloxane, cyclopentadimethylsiloxane, cyclohexadimethylsiloxane, cyclocopolymer of dimethylsiloxane/methyloctylsiloxane, hexylheptamethyltrisiloxane or octylheptamethyltrisiloxane.

Another embodiment of the instant invention is a coated pigment particle comprising a pigment particle and the coating on said pigment particle wherein said coating comprises one or more silicone compounds having formula (1), (2), and (3) wherein the silicone compound of formula (3) is defined as:

x is an integer from 1 to about 500.

Another embodiment of the instant invention is a coated pigment particle comprising a pigment particle and the coating on said pigment particle wherein said coating comprises one or more silicone compounds having formula (1), (2), and (3) wherein the silicone compound of formula (3) is defined as:

x is an integer from 1 to about 50.

Another embodiment of the instant invention is a coated pigment particle comprising a pigment particle and the coating on said pigment particle wherein said coating comprises one or more silicone compounds having formula (1), (2), and (3) wherein the silicone compound of formula (3) is dimethylpolysiloxane.

Another embodiment of the instant invention is a coated pigment particle comprising a pigment particle and the coating on said pigment particle wherein said coating comprises cetyldimethicone and silicone compounds having formula (2) and/or (3) wherein the silicone compound of formula (2) is defined as:

R5 is C1 to C6 branched or straight chain alkyl; R6 is C1 to C6 branched or straight chain alkyl; R7 is C1 to C6 branched or straight chain alkyl; R8 is C1 to C16 branched or straight chain alkyl; a is from 1 to about 25; b is from 1 to about 25; the sum of a+b is from 2 to about 25.

Another embodiment of the instant invention is a coated pigment particle comprising a pigment particle and the coating on said pigment particle wherein said coating comprises cetyldimethicone and silicone compounds having formula (2) and/or (3) wherein the silicone compound of formula (2) is defined as:

R5 is methyl; R6 is methyl; R7 is methyl; R8 is C1 to C8 straight chain alkyl; a is from 1 to about 9; b is from 1 to about 9; the sum of a+b is from 3 to about 9.

Another embodiment of the instant invention is a coated pigment particle comprising a pigment particle and the coating on said pigment particle wherein said coating comprises cetyldimethicone and silicone compounds having formula (2) and/or (3) wherein the silicone compound of formula (2) is, for example, cyclotetradimethylsiloxane, cyclopentadimethylsiloxane, cyclohexadimethylsiloxane, cyclocopolymer of dimethylsiloxane/methyloctylsiloxane, hexylheptamethyltrisiloxane or octylheptamethyltrisiloxane.

Another embodiment of the instant invention is a coated pigment particle comprising a pigment particle and the coating on said pigment particle wherein said coating comprises cetyldimethicone and silicone compounds having formula (2) and/or (3) wherein the silicone compound of formula (3) is defined as:

x is an integer from 1 to about 500.

Another embodiment of the instant invention is a coated pigment particle comprising a pigment particle and the coating on said pigment particle wherein said coating comprises cetyldimethicone and silicone compounds having formula (2) and/or (3) wherein the silicone compound of formula (3) is defined as:

x is an integer from 1 to about 50.

Another embodiment of the instant invention is a coated pigment particle comprising a pigment particle and the coating on said pigment particle wherein said coating comprises cetyldimethicone and silicone compounds having formula (2) and/or (3) wherein the silicone compound of formula (3) is dimethylpolysiloxane.

Another embodiment of the instant invention is a coated pigment particle comprising a pigment particle and the coating on said pigment particle wherein the weight ratio of formula (1) to formula (2) and/or formula 3 is from about 1000:1 to about 1:1000.

Another embodiment of the instant invention is a coated pigment particle comprising a pigment particle and the coating on said pigment particle wherein the weight ratio of formula (1) to formula (2) and/or formula 3 is from about 100:1 to about 1:100.

Another embodiment of the instant invention is a coated pigment particle comprising a pigment particle and the coating on said pigment particle wherein the weight ratio of formula (1) to formula (2) and/or formula 3 is from about 10:1 to about 1:10.

Another embodiment of the instant invention is a coated pigment particle comprising a pigment particle and the coating on said pigment particle wherein the weight percent of the coating on the particles is in the range from about 0.1 percent to about 90 percent based on the weight of the coated pigment particle. An effective amount of the silicone compounds is in the range from about 0.1 percent to about 30 percent based on the weight of the coated pigment particle. An effective amount of the silicone compounds is in the range from about 1 percent to about 15 percent based on the weight of the coated pigment particle. An effective amount of the silicone compounds is in the range from about 3 percent to about 10 percent based on the weight of the coated pigment particle

Although there are no critical limitations to the sizes of the pigment particles, preferably pigment particles having an average size from about 0.01 to about 500 micrometers, more preferably having an average size of about 1 to about 200 micrometers, most preferably having an average size from about 5 to about 150 micrometers are coated with the silicone compounds according to formula (1), (2), or (3) of the instant invention.

Another embodiment of the instant invention is a coated pigment particle comprising a pigment particle and the coating on said pigment particle wherein the pigment particle is selected from the group consisting of inorganic pigments, metal oxides and hydroxides, mica, organic pigments, pearlescent pigments, mineral silicates, porous materials, carbons, and interference pigments.

Another embodiment of the instant invention is a coated pigment particle comprising a pigment particle and the coating on said pigment particle wherein the pigment particle is selected from the group consisting of inorganic pigments, metal oxides and hydroxides, mica, organic pigments, pearlescent pigments, and interference pigments.

Another embodiment of the instant invention is a coated pigment particle comprising a pigment particle and the coating on said pigment particle wherein the pigment particle is selected from the group consisting of inorganic pigments, organic pigments, pearlescent pigments, and interference pigments.

Another embodiment of the instant invention is a coated pigment particle comprising a pigment particle and the coating on said pigment particle wherein the pigment particle is pearlescent pigments or interference pigments.

Another embodiment of the instant invention is more than one or a blend of pigment particles some of which are modified according to the instant invention. For example, a pearlescent pigment modified according to the instant method and an unmodified organic pigment. For example, a pearlescent pigment modified according to the instant method and an interference pigment modified according to the instant method. Still further, a blend of two or more pigments some of which are modified according to the instant invention.

Another embodiment of the instant invention is a blend of pigment particles some of which are modified according to the instant invention wherein the weight ratio one pigment to the other is from about 1000:1 to about 1:1000.

Another embodiment of the instant invention is a blend of pigment particles some of which are modified according to the instant invention wherein the weight ratio one pigment to the other is from about 100:1 to about 1:100.

Another embodiment of the instant invention is a blend of pigment particles some of which are modified according to the instant invention wherein the weight ratio one pigment to the other is from about 10:1 to about 1:10.

When a blend of two or more pigments are used some of which are modified according to the instant invention an appropriate ratio of pigment weights is used to bring about the desired color or shade or coloristic effect.

According to the instant invention, various kinds of pigment particle surfaces can be coated the thereon with a silicone compound. Examples of pigment particles that can be treated according to the instant invention, but is not limited to, include inorganic pigments, metal oxides and hydroxides, mica, organic pigments, pearlescent pigments, mineral silicates, porous materials, carbons, interference pigments, and the like.

Examples of the inorganic pigments capable of being modified according to the present invention are ultramarine blue, ultramarine violet, Prussian blue, manganese violet, titanium-coated mica, bismuth oxychloride, iron oxides, iron hydroxide, titanium dioxide, titanium lower oxides, chromium hydroxide and oxides, and carbon based pigments (e.g. Carbon Black). Of these pigments, ultramarine blue and Prussian blue are typically modified according to the present invention.

As well-known in the art, ultramarine blue (i.e., sodium aluminum silicate containing sulfur) is generally represented by Na6-9Al6Si6O24S2-4, and conventionally and widely used as a blue inorganic pigment in various fields (e.g., coating compositions, paints, inks, cosmetics, and detergents). Ultramarine blue has hydrophilicity and is stable up to a temperature of about 250 C in an air atmosphere. However, ultramarine blue is not stable against an acid, although it is generally stable against an alkali. For example, ultramarine blue is gradually decomposed to generate hydrogen sulfide under an acidic condition (e.g., in the presence of a radical sulfate). As a result, the resultant ultramarine blue is discolored and becomes white. Obviously, the generation of hydrogen sulfide is especially not preferable in the fields of, for example, cosmetics. Furthermore, ultramarine blue is likely to generate hydrogen sulfide from a mechanical shearing force (e.g., grinding) or heating. Although various attempts have been made to obviate these disadvantages, as shown in Japanese Unexamined Patent Publication (Kokai) No. 54-95632 and Japanese Examined Patent Publication (Kokoku) No. 50-27483, the effects thereof are not sufficient from a practical point of view.

However, when ultramarine blue is modified with the silicone compound of formula (1), (2), and/or (3) according to the present invention, the generation of hydrogen sulfide under an acidic condition or at an elevated temperature or by a mechanical shearing force can be effectively prevented, and the decomposing action thereof against, for example, perfumes, can be suppressed. Thus, when the ultramarine blue modified with the silicone compound of formula (1), (2), and/or (3) is used under an acidic condition, no substantial deterioration occurs in aluminum or silver containers or in cosmetics. Furthermore, since the modified ultramarine blue is coated with the silicone compound layer, hydrophobicity is exhibited and the wettability is improved. Thus, the modified ultramarine blue can be formulated into an oil phase in an emulsion system.

The above-mentioned inherent disadvantages of ultramarine blue are believed to be caused by the presence of sulfur on the surfaces of ultramarine blue particles (i.e., surface sulfur). This surface sulfur is an active radical-type sulfur present on the surface of the crystalline lattice of ultramarine blue, which is likely to be susceptible to, for example, an acid, thermal, or mechanical shearing force action. However, according to the present invention, the surfaces of ultramarine blue particles are covered by the silicone compound layer to stabilize the ultramarine blue particles. Furthermore, since the silicone compound layer has a high transparency, there is no substantial difference between the unmodified and modified ultramarine blue particles.

Ultramarine blue to be modified according to the present invention can be any conventional ultramarine blue particles having a size of, for example, 0.1 to 100 micrometers. The amount of the silicone compound present in the stabilized ultramarine blue powder particle is typically about 0.1% to 20% by weight, preferably 0.2% to 10% by weight based on the weight of the particle, depending upon the surface area and activity of the particle. The ultramarine blue particles may be dried prior to the treatment, if desired. Furthermore, conventional composite powder particles of, for example, plastics or metal oxides coated with ultramarine blue, also can be treated according to the present invention.

As well-known in the art, Prussian blue (i.e., ferric ferrocyanide) is generally represented by MFe[Fe(CN)6], wherein M represents K, NH4, or Na, and is conventionally and widely used as a blue inorganic pigment having a large coloring power in various fields (e.g., coating compositions, paints, cosmetics). However, Prussian blue has a poor alkaline resistance although the acid resistance is strong. Furthermore, Prussian blue is not stable against heating which causes the decomposition or a discoloration to dark brown. Further, upon heating, Prussian blue is susceptible to reduction and tends to cause the deterioration of co-existence substances (e.g., perfumes).

However, when Prussian blue is modified with the silicone compound of formula (1), (2), and/or (3) according to the present invention, the above-mentioned disadvantages of conventional Prussian blue can be effectively eliminated. Thus, the modified Prussian blue is formulated into compositions such as cosmetics, pharmaceutical compositions, and the stability thereof is remarkably improved because an undesirable interaction thereof with other ingredients (e.g., perfumes) can be eliminated. Furthermore, since the silicone compound layer is highly transparent, no substantial difference is observed in color between the unmodified and the modified Prussian blue particles.

The Prussian blue powder particles to be modified according to the present invention can be any conventional Prussian blue powder particles, preferably having a size of 0.01 to 100 micrometers, more preferably 0.05 to 0.1 micrometers. The amount of silicone compound coated on the surface of the modified Prussian blue particles is typically 0.5% to 40% by weight, more preferably 5% to 30% by weight, depending upon the surface area of the particle.

Examples of the metal oxides and hydroxides capable of being modified according to the present invention are magnesium oxide, magnesium hydroxide, calcium oxide, calcium hydroxides, aluminum oxide, aluminum hydroxide, silica, iron oxides (.alpha-Fe2O3, gamma-Fe2O3, Fe3O4, FeO), iron hydroxides, titanium dioxide, titanium lower oxides, zirconium oxide, chromium oxides, chromium hydroxides, manganese oxides, cobalt oxides, nickel oxides, and zinc oxides. These oxides and hydroxides may be used alone or in any mixture thereof. Furthermore, composite oxides and composite hydroxides such as iron titanate, cobalt titanate, and cobalt aluminate also can be used in the present invention. Composite materials comprising metal oxides or hydroxides coated on the core materials (e.g., titanium oxides coated mica, iron oxides coated nylon) can also be used in the present invention.

Although there are no critical limitations to the sizes of the metal oxide or hydroxide powder particles, metal oxides or hydroxides having a size of 0.001 to 500 micrometers, preferably 0.01 to 200 micrometers can be preferably coated with the silicone compound without causing agglomeration of the powder particles. The preferable coating amount of the silicone compound is 0.1% to 20% by weight, more preferably 0.2% to 10% by weight, depending upon the kinds and surface area of the metal oxides or hydroxides.

As well-known in the art, metal oxides and hydroxides are conventionally and widely used as, for example, a colorant in various application fields (e.g., coating compositions, paints, cosmetics, inks) or as a magnetic material. However, conventional metal oxides and hydroxides generally have hydrophilicity and, therefore, have a poor dispersibility thereof in oils or organic solvents. Furthermore, metal oxides and hydroxides have catalyst activities and, therefore, deteriorate co-existing substances such as fats and oils and perfumes or cause discoloration due to the surface activities.

On the other hand, when metal oxides and hydroxides are modified with the silicone compounds according to formula (1), (2), and/or (3) according to the present invention, the resultant metal oxides and hydroxides are uniformly covered with the silicone compound layer over the entire surfaces thereof and, therefore, the metal oxides and hydroxides are stabilized so that they do not interact with other agents and do not deteriorate perfumes and the like. Thus, when the modified metal oxides and hydroxides are formulated into various compositions (e.g., coating compositions, paints, cosmetics, pharmaceutical compositions), the stability thereof with the lapse of time is remarkably improved. Furthermore, since the silicone compound layer is very thin and is transparent, a color difference between the untreated and treated metal oxides and hydroxides is not observed and the magnetic properties of the metal oxides and hydroxides, if any, are not adversely affected. Thus, the modified gamma-Fe2O3 or Co-gamma-Fe2O3 may be advantageously used in the production of magnetic recording materials.

Examples of mica capable of being modified according to the present invention are muscovite, phlogopite, biotite, sericite, lepidolite, paragonite and artificial or synthetic mica having a fluorine atom substituted for the hydroxyl group of natural mica as well as baked or calcined products thereof. These mica pigments may be used alone or in any mixture thereof.

Although raw mica ores may be modified according to the present invention, mica having a size of 0.5 to 200 micrometers may be preferably modified with the silicone compounds of formula (1), (2), and/or (3) according to the present invention. However, mica can be modified according to the present invention after the cleaving thereof to a thin form (e.g., flakes). Although there are no critical limitations to the size of the mica, the preferable coating amount of the silicone compound of formula (1), (2), and/or (3) is 0.1% to 20% by weight, more preferably 0.2% to 10% by weight, in the case of the inherent mica. However, when the excess amount of the silicone compounds of formula (1), (2), and/or (3) in the form of vapor is introduced, the mica is expanded due to the occurrence of the cleavage of the mica and, therefore, up to 20% to 90% by weight of the silicone compound may be used.

As well-known in the art, mica is conventionally and widely used as a filler or additive in, for example, coating compositions, inks, and cosmetics, as well as plastics and rubbers. However, mica generally has hydrophilicity and, therefore, has a poor dispersibility in oils and organic solvents. Furthermore, when mica is kneaded to plastics and rubbers, mica is likely to cause aggregation and uniform kneading is difficult.

On the other hand, when mica is modified with the silicone compound of formula (1), (2), and/or (3) according to the present invention, the modified mica is uniformly covered with the thin silicone compound layer over the entire surfaces thereof and, therefore, the mica is stabilized. Accordingly, when the modified mica is incorporated into compositions, the mica does not decompose or deteriorate co-existing substances (e.g., perfumes) and, therefore, the stability of the cosmetics and pharmaceutical compositions with the lapse of time is remarkably improved. Furthermore, since the silicone compound layer is thin and transparent, there are no substantial differences in color between the untreated and modified mica. In addition, the modified mica exhibits hydrophobicity and can be formulated into an oil phase in the case of emulsions.

Examples of the organic pigments capable of being modified with the silicone compounds of formula (1), (2), or (3) according to the present invention are C.I. 15850, C.I. 15850:1, C.I. 15585:1, C.I. 15630, C.I. 15880:1, C.I. 73360, C.I. 12085, C.I. 15865:2, C.I. 12075, C.I. 21110, C.I. 21095, and C.I. 11680, C.I. 74160 and zirconium, barium, or aluminum lakes of C.I. 45430, C.I. 45410, C.I. 45100, C.I. 17200, C.I. 45380, C.I. 45190, C.I. 14700, C.I. 15510, C.I. 19140, C.I. 15985, C.I. 45350, C.I. 47005, C.I. 42053, C.I. 42090.

C.I. means Colour Index as compiled by the by The Society of Dyers and Colourists and The American Association of Textile Chemists and Colourists.

The range of interesting organic pigments may additionally comprise monoazo, disazo, naphthol, dioxazone, azomethin, azocondensation, metal complex, nitro, perinone, quinoline, anthraquinone, benzimidozolone, isoindoline, isoindolinone, triarylmethane, quinacridone, hydroxyanthraquinone, aminoanthraquinone, anthrapyrimidine, indanthrone, flavanthrone, pyranthrone, anthantrone, isoviolanthrone, diketopyrrolopyrrole, carbazole, indigo or thiolndigo pigments.

Mixtures of the organic pigments may be used.

Examples of these pigments and also further pigments can be found in the monograph: W. Herbst, K. Hunger “Industrielle Organische Pigmente,” 2.sup.nd edition, 1995, VCH Verlagsgesellschaft.

Preferred pigments for the application of the present invention are mono- or disazo pigments, preferably mono- or diarylides, or metal complexes, preferably a copper phthalocyanine pigment, or naphthol pigments, preferably .beta.-naphthol or a .beta.-oxynaphthoic acid (BONA) pigments, or quinacridones or indanthrones.

More preferably, the following organic pigments are used inventively:

Mono- or diarylide yellow pigments selected from C.I. Pigment Yellow 1, 2, 10, 12, 13, 14, 17, 61, 62, 63, 64, 65, 73, 74, 75, 83, 127, 168, 174, 176, 188 and 191.

Monoarylide yellow pigments selected from C.I. Pigment Yellow 1, C.I. Pigment Yellow 2, C.I. Pigment Yellow 65, C.I. Pigment Yellow 73, C.I. Pigment Yellow 74 and C.I. Yellow 75.

Diarylide yellow pigments selected from C.I. Pigment Yellow 12, C.I. Pigment Yellow 13, C.I. Pigment Yellow 14, C.I. Pigment Yellow 17 and C.I. Pigment Yellow 83.

Disazo orange pigments selected from C.I. Pigment Orange 16 and C.I. Pigment Orange 34.

Naphthol red pigments selected from C.I. Pigment Red 48:1, C.I. Pigment Red 48:2, C.I. Pigment Red 48:3, C.I. Pigment Red 48:4, C.I. Pigment Red 48:5, C.I. Pigment Red 49:1, C.I. Pigment Red 52:1, C.I. Pigment Red 52:2, C.I. Pigment Red 52:3, C.1 Pigment Red 53:1, C.I. Pigment Red 53:2, C.I. Pigment Red 53:3, C.I. Pigment Red 57:1, C.I. Pigment Red 57:2, C.I. Pigment Red 58:2, C.I. Pigment Red 58:4, C.I. Pigment Red 63:1 and C.I. Pigment Red 64:1.

Quinacridone red pigments such as C.I. Pigment Red 202.

Blue or green copper phthalocyanine pigments selected from C.I. Pigment Blue 15:1, C.I. Pigment Blue 15:2, C.I. Pigment Blue 15:3, C.I. Pigment Blue 15:4, C.I. Pigment Green 7 and C.I. Pigment Green 36.

Blue indanthrone pigments such as C.I. Pigment Blue 60.

The surfaces of these organic pigments may be treated additionally with, for example, rosins.

Although these are no critical limitations to the sizes of the organic pigments, the organic pigments having a size of 0.05 to 100 micrometers may be preferably coated or covered with the silicone compounds of formula (1), (2), and/or (3). The preferable coating amount of the silicone compound is 0.1% to 20% by weight, more preferably 0.2% to 10% by weight, depending upon the kinds and surface area of the organic pigments.

As well-known in the art, organic pigments are conventionally and widely used as a colorant in, for example, coating compositions, inks, and cosmetics, as well as plastics and rubbers. However, conventional organic pigments or the surface-treated products thereof cause the decomposition or deterioration of co-existing substances when formulated into compositions due to the interaction thereof, based on the presence of the active sites on the surface thereof, with the co-existing substances. Furthermore, certain organic pigments cause discoloration due to, for example, the absorption or desorption of the water of crystallization.

On the other hand, when organic pigments are modified with the silicone compound of formula (1), (2), and/or (3) according to the present invention, the modified organic pigments have the uniform silicone compound layer covering the entire surface thereof. Thus, the modified organic pigments become stable and have no interaction activities against the co-existing substances. Accordingly, when the modified organic pigments are formulated into compositions, the stability of the compositions with the lapse of time is remarkably improved. Furthermore, since the silicone compound layer is thin and highly transparent, there is no difference in the color of the organic pigments before and after the treatment. In addition, the dispersibility thereof in vehicles is also improved.

Examples of pearlescent pigments (or nacreous pigments) are mica-titanium composite materials containing, as a titanium component, titanium dioxide, titanium lower oxides, titanium oxynitride, mica-iron oxide composite materials, bismuth oxychloride, and guanine. The mica-titanium composite materials may be mixed with colored pigments such as iron oxides, Prussian blue, chromium oxide, carbon black, and carmine. These pearlescent pigments may be used alone or in any mixture thereof.

Although there are no critical limitations to the sizes of the pearlescent pigments, the pearlescent pigments having a size of 1 to 200 micrometers can be preferably coated or covered with the uniform silicone compound layer of formula (1), (2), and/or (3) according to the instant invention over the entire surface thereof. Note, in the case of the pearlescent pigments, the powder particles are preferably in the form of a flat shape (e.g., flakes). The preferable coating amount of the silicone compound is approximately 0.01% to 20% by weight.

As well-known in the art, pearlescent pigments are widely used in, for example, coating compositions, inks, cosmetics, plastics, ceramics, decorating, daily necessities, and fiber or textile products. Mica is generally formulated into these compositions, together with colored pigments, to exhibit various colored appearance. However, when mica composite materials are used together with other substances, the composite materials themselves are deteriorated or cause the discoloration or deterioration of co-existing substances. Another problem with pearlescent pigments is that these pigments particles can't be ground into the oil phase like other inorganic or organic pigments, as the distinct pearlescence, luster and sparkling effects are dependent upon particle size, which in turn would be changed by the grinding process. For cosmetic formulations delivery of the color effect to the skin is essential. Pearlescent pigments however do typically have very low affinity to skin, making formulation of makeup products more demanding and reducing durability of the color effect on skin. Additionally, their dusting properties make pearl pigments difficult to handle and big particle size pearls leave an uncomfortable, raspy feel on the skin.

On the other hand, according to the present invention, when the pearlescent pigments are uniformly coated or covered with the silicone compound film, the resultant pearlescent pigments are stabilized and do not cause the deterioration or decomposition of co-existing substances. They can be easily stirred into oil phase of formulations without heavy effects on viscosity and they adhere to skin better, improving overall application properties in cosmetics. Another advantage is that the pearlescent pigments coated according to present invention exhibit an excellent skin feel, providing smooth and silky texture even to high particle size pigments. We also found that the color intensity increases and metal-like shine is achieved. An additional benefit is the significantly reduced dusting issue.

Examples of the mineral silicates capable of being modified according to the present invention are phyllosilicates and tectosilicates such as pyrophyllite, talc, chlorite, chrysotile, antigorite, lizardite, kaolinite, dickite, nacrite, halloysite, montmorillonite, nontronite, saponite, sauconite, and bentonite; natrolites such as natrolite, mesolite, scolecite, and thomsonite; heulandites such as heulandite, stilbite, epistibite; and zeolites such as analcite, harmotone, phillipsite, chabazite, and gmelinite. These silicate minerals may be used alone or in combination thereof. The phyllosilicates may have organic cations at the interface of the layers thereof or may be substituted with alkali metal or alkaline earth metal ions. The tectosilicates may include metallic ions in the fine pores thereof.

Although there are no critical limitations to the sizes of the silicate minerals, preferably silicate minerals having a size of 0.01 to 100 micrometers, more preferably 0.1 to 30 micrometers are modified with the silicone compounds according to formula (1), (2), and/or (3) of the instant invention. The preferable coating amount of the silicone compound is 0.1% to 20% by weight, more preferably 0.2% to 5.0% by weight, depending upon the kinds and surface area of the silicate mineral particles.

As well-known in the art, conventional silicate minerals optionally coated with certain silicone resins are widely used as an electric insulating material, filler, and additives in various fields (e.g., pharmaceutical compositions, ceramics, paper, rubbers, inks, cosmetics, and coating compositions). However, conventional silicate minerals unpreferably cause a deterioration of co-existing substances (e.g., perfumes, oils and fats, resins) when formulated into, for example, cosmetics and coating compositions.

On the other hand, according to the present invention, the silicate minerals can be stabilized because the surfaces of the silicate mineral particles can be uniformly coated or covered with a thin silicone compound layer. As a result, the modified silicate minerals do not deteriorate the co-existing substances when formulated therewith into compositions.

Examples of the porous materials capable of being modified with the silicone compounds of formula (1), (2), or (3) according to the present invention are the above-mentioned silicate minerals; the above-mentioned mica; the above-mentioned metal oxides; KAl2(Al,Si3)O10F2, KMg(Al,Si3)O10F2, and K(Mg,Fe3)(Al,Si3)O10F2; carbonate minerals such as CaCO3, MgCO3, FeCO3, MnCO3, ZnCO3, CaMg(CO3)2, Cu(OH)2CO3, and Cu3(OH)2(CO3)2; sulfate minerals such as BaSO4, SrSO4, PbSO4, CaSO4, CaSO4.2H2O, CaSO4.5H2O, Cu4SO4(OH)6, KA13(OH)6(SO4)2, and KFe3(OH)6(SO4)2; phosphate minerals such as YPO4, (Ce, La)PO4Fe3(PO4)2.8H2O, Ca5(PO4)3F, Ca5(PO4)3Cl, Ca5(PO4)3OH, and Ca5(PO4, CO3OH)3(F, OH); and metal nitrides such as titanium nitride, boron nitride, and chromium nitride. These materials may be used alone or in any mixture thereof. Furthermore these porous materials may be modified after granulation or molding, followed by baking or calcining. Furthermore, celluloses, fibers, and synthetic resins may be modified with the silicone compounds of formula (1), (2), and/or (3) according to the present invention.

Although there are no critical limitations to the sizes of the porous materials, the porous materials having a size of 10 mm or less, more preferably 3 micrometers to 10 mm, may be preferably coated with the silicone compounds according to the instant invention. The preferable coating amount of the silicone compound is approximately 0.01 to 20% by weight, depending upon the kinds and surface areas of the porous materials.

According to the present invention, the silicone compound deposited on the surface of the porous material and on the surface of the micropores of the porous material is sufficient to decrease any detrimental surface activity of the said porous material. Thus, the modified porous materials do not cause a deterioration of substances such as pharmaceutical agents and perfumes. Accordingly, the modified porous materials may be advantageously used in, for example, perfumes, pharmaceutical compositions, toys, artificial organs, artificial bones, and ceramics.

Examples of the carbons capable of being modified with the silicone compounds of formula (1), (2), and/or (3) are the activated carbon and carbon black conventionally used in, for example, coating compositions, inks, tires, and cosmetics.

Although there are no critical limitations to the sizes of the carbon powder particles, preferably carbon powder particles having a size of 0.001 to 200 micrometers, more preferably 0.01 to 100 micrometers are modified with the silicone compounds according to the present invention. The preferable coating amount of the silicone compound is approximately 0.1 to 50% by weight, more preferably 1% to 30% by weight, depending upon the kinds and surface areas of the carbon powder particles.

As well-known in the art, carbon powder particles are widely used as a colorant in, for example, coating compositions, inks, and cosmetics. However, since carbon is generally hydrophilic, the dispersibility thereof in oils and organic solvents is not good. Furthermore, carbon tends to deteriorate co-existing substances such as oil and fats and perfumes and to adsorb valuable agents thereon.

On the other hand, according to the present invention, the silicone compound deposited on the surface of the carbon powder particles forms a uniform silicone compound layer on the entire surface thereof. As a result, the surface activity of the carbon disappears, and the modified carbon becomes hydrophobic or lipophilic. Accordingly, the modified carbon may be advantageously formulated into, for example, coating compositions, inks, and cosmetics, without causing any deterioration of co-existing substances such as oil and fats and perfumes.

According to the instant invention, interference pigments are modified with the silicone compounds of formula (1), (2), and/or (3). An interference pigment is a pigment with pearl gloss prepared by coating the surface of a particle substrate material with a thin film. The particle substrate material is generally platelet in shape. The thin film is a transparent or semitransparent material having a high refractive index. The high refractive index material shows a pearl gloss resulting from mutual interfering action between reflection and incident light from the platelet substrate/coating layer interface and reflection of incident light from the surface of the coating layer.

The interference pigments of the present invention are platelet particulates. The platelet particulates preferably have a thickness of no more than about 5 micrometers, more preferably no more than about 2 micrometers, still more preferably no more than about 1 micrometer. The platelet particulates preferably have a thickness of at least about 0.02 micrometers, more preferably at least about 0.05 micrometers, even more preferably at least about 0.1 micrometers, and still more preferably at least about 0.2 micrometers.

The particle size determines the opacity and luster. The particle size is determined by measuring the diameter thickness of the particulate material. The term “diameter” as used herein, means the largest distance across the major axis of the particulate material. Diameter can be determined by any suitable method known in the art, such as particle size analyzer Mastersizer 2000 manufactured by Malvern Instruments. The interference pigments preferably have an average diameter not greater than about 200 micrometers, more preferably not greater than 150 micrometers. The interference pigments preferably have a diameter of at least about 0.1 micrometer, more preferably at least about 1.0 micrometer, even more preferably at least about 2.0 micrometer, and still more preferably at least about 5.0 micrometer.

The interference pigments comprise a multilayer structure. The center of the particulates is a flat substrate with a refractive index (R1) normally below 1.8. A wide variety of particle substrates are useful herein. Nonlimiting examples are natural mica, synthetic mica, graphite, talc, kaolin, alumina flake, bismuth oxychloride, silica flake, glass flake, ceramics, titanium dioxide, CaSO4, CaCO3, BaSO4, borosilicate and mixtures thereof, preferably mica, silica and alumina flakes.

A layer of thin film or a multiple layer of thin films are coated on the surface of a substrate described above. The thin films are made of highly refractive materials. The refractive index of these materials is normally above 1.8.

A wide variety of thin films are useful herein. Nonlimiting examples are TiO2, Fe2O3, SnO2, Cr2O3, ZnO, ZnS, SnO, ZrO2, CaF2, Al₂O3, BiOCl, and mixtures thereof or in the form of separate layers, preferably TiO2, Fe2O3, Cr2O3, and SnO2. For the multiple layer structures, the thin films can be consisted of all high refractive index materials or alternation of thin films with high and low R1 materials with the high R1 film as the top layer.

The interference pigment color is a function of the thickness of thin film, the thickness for a specific color may be different for different materials. For TiO2, a layer of 40 nm to 60 nm or a whole number multiple thereof gives silver color, 60 nm to 80 nm yellow color, 80 nm to 100 nm red color, 100 nm to 130 nm blue color, 130 nm to 160 nm green color. In addition to the interference color, other transparent absorption pigments can be precipitated on top of or simultaneously with the TiO2 layer. Common materials are red or black iron oxide, ferric ferrocyanide, chromium oxide or carmine. It is found that the color of the interference pigment in addition to its brightness has a significant influence on human perception of skin tone. In general, preferred colors are silver, gold, red, green and mixtures thereof.

Nonlimiting examples of the interference pigments useful herein include those supplied by Persperse, Inc. under the trade name PRESTIGE®, FLONAC®; supplied by EMD Chemicals, Inc. under the trade name TIMIRON®, COLORONA®, DICHRONA® and XIRONA®; supplied by Engelhard Co. under the trade name FLAMENCO®, TIMICA®, DUOCHROME®; and supplied by Ciba under the trade name CALISHA®

As with mica composite materials, the interference pigments themselves may deteriorate or cause the discoloration or deterioration of co-existing substances. The interference pigments can't be ground into the oil phase like other inorganic or organic pigments, as the distinct highlights, luster and sparkling effects are depending on particle size, which in turn would be changed by the grinding process. For cosmetic formulations delivery of the color effect to the skin is essential. Interference pigments however do typically have very low affinity to skin, making formulation of makeup products more demanding and reducing durability of the color effect on skin. Their dusting properties make interference pigments difficult to handle.

On the other hand, according to the present invention, when the interference pigments are uniformly coated or covered with the silicone compound film, the resultant interference pigments are stabilized and do not cause the deterioration or decomposition of co-existing substances. They can be easily stirred into oil phase of formulations without heavy effects on viscosity and they adhere to skin better, provide smooth and silky texture even to big particle size pigments and impart an almost metallic shine, thus improving overall application properties in cosmetics. An additional benefit is the significantly reduced dusting issue.

Another embodiment of the instant invention is personal care compositions comprising pigment particles that are modified according to the instant method and/or a coated pigment particle according to the instant invention.

The modified or coated pigment particles of the personal care compositions comprises from about 100 weight percent of the composition; preferably no more than about 50 weight percent of the personal care composition; more preferably no more than about 25 weight percent of the personal care composition even more preferably no more than about 7 weight percent; and still more preferably no more than about 5 weight percent. The modified pigment particles of the personal care composition preferably comprises at least about 0.001 weight percent of the personal care composition, more preferably at least about 0.01 weight percent, even more preferably at least about 0.1 weight percent, and still more preferably at least about 0.2 by weight of the composition.

Personal care compositions according to the invention may be contained in a wide variety of personal care preparations. Especially the following preparations, for example, come into consideration:
shaving preparations, e.g. shaving soap, foaming shaving creams, non-foaming shaving creams, foams and gels, preshave preparations for dry shaving, aftershaves or aftershave lotions;
skin-care preparations, e.g. skin emulsions, multi-emulsions or skin oils and body powders;
cosmetic personal care preparations, e.g. facial make-up in the form of lipsticks, eye shadow, eye liners, liquid make-up, day creams or powders, facial lotions, foundations, creams and powders (loose or pressed);
light-protective preparations, such as sun tan lotions, creams and oils, sun blocks and pretanning preparations;
manicure preparations, e.g. nail polishes, nail enamels, enamel removers, nail treatments
deodorants, e.g. deodorant sprays, pump-action sprays, deodorant gels, sticks or roll-ons;
antiperspirants, such as antiperspirant sticks, creams or roll-ons; and
solid/liquid personal cleaning products, such as soap, cleansers, shampoo, conditioners, hair treatments.

Another embodiment of the instant invention is a personal care composition which is formulated as a water-in-oil or oil-in-water emulsion, as an alcoholic or alcohol-containing formulation, as a vesicular dispersion of an ionic or non-ionic amphiphilic lipid, as a gel, or a solid stick as an aqueous or non-aqueous system.

Another embodiment of the instant invention is a personal care composition which further comprises at least one further constituent selected from the group consisting of sequestering agents, non-encapsulated colorings, perfumes, thickening or solidifying (consistency regulator) agents, emollients, UV absorbers, surfactants, bleaching agents, skin-protective agents, antioxidants and preservatives.

Another embodiment of the instant invention is a personal care composition wherein the personal care or cosmetic composition comprises a blend of modified pigment particles that are individually provided in a single matrix material.

Depending upon the form of the personal care composition or preparation, it will comprise, in addition to the modified pigment particles of the instant invention, further constituents, for example sequestering agents, additional non-encapsulated colorings, perfumes, thickening or solidifying (consistency regulator) agents, emollients, UV absorbers, skin-protective agents, antioxidants, preservatives, and the like.

The personal care compositions of the present invention may contain one or more additional skin care or hair care components. In a preferred embodiment, where the composition is to be in contact with human keratinous tissue, the additional components should be suitable for application to keratinous tissue, that is, when incorporated into the composition they are suitable for use in contact with human keratinous tissue without undue toxicity, incompatibility, instability, allergic response, and the like within the scope of sound medical judgment.

The CTFA Cosmetic Ingredient Handbook, Second Edition (1992) describes a wide variety of non-limiting cosmetic and pharmaceutical ingredients commonly used in the personal care industry, which are suitable for use in the personal care compositions of the present invention.

The present invention may optionally comprise an oil structurant. The structurant can provide the dispersed phase with the correct rheological properties. This can aid in providing effective deposition and retention to the skin, the structured oil or oil phase should have a viscosity in the range of 100 to about 200,000 poise measured at 1 Sec-1, preferably 200 to about 100,000 poise, and most preferably 200 to about 50,000 poise. The amount of structurant required to produce this viscosity will vary depending on the oil and the structurant, but in general, the structurant will preferably be less than 75 weight percent of the dispersed oil phase, more preferably less than 50 weight percent, and still more preferably less than 35 weight percent of the dispersed oil phase.

The structurant can be either an organic or inorganic structurant. Examples of organic thickeners suitable for the invention are solid fatty acid esters, natural or modified fats, fatty acid, fatty amine, fatty alcohol, natural and synthetic waxes, and petrolatum, and the block copolymers sold under the name KRATON by Shell. Inorganic structuring agents include hydrophobically modified silica or hydrophobically modified clay. Nonlimiting examples of inorganic structurants are BENTONE 27V, BENTONE 38V or BENTONE GEL MIO V from Rheox; and CAB-O-SIL TS720 or CAB-O-SIL M5 from Cabot Corporation.

Structurants meeting the above requirements with the selected skin compatible oil can form 3-dimensional network to build up the viscosity of the selected oils. It has been found that such structured oil phases, i.e., built with the 3-dimensional network, are extremely desirable for use as wet-skin treatment compositions used in bathing. These structured oils can deposit and be retained very effectively on wet skin and retained after rinsing and drying to provide long-lasting after wash skin benefit without causing a too oily/greasy wet and dry feel. It is believed that the highly desirable in-use and after-use properties of such structured oils are due to their shear thinning rheological properties and the weak structure of the network. Due to its high low-shear viscosity, the 3-dimensional network structured oil can stick and retain well on the skin during application of the skin conditioner. After being deposited on the skin, the network yields easily during rubbing due to the weak structuring of the crystal network and its lower high-shear viscosity.

A wide variety of surfactants can be useful herein, both for emulsification of the dispersed phase as well as to provide acceptable spreading and in use properties for non-lathering systems. For cleansing applications, the surfactant phase also serves to clean the skin and provide an acceptable amount of lather for the user. The composition preferably contains no more than about 50 weight percent of a surfactant, more preferably no more than about 30 weight percent, still more preferably no more than about 15 weight percent, and even more preferably no more than about 5 weight percent of a surfactant. The composition preferably contains at least about 5 weight percent of a surfactant, more preferably at least about 3 weight percent, still more preferably at least about 1 weight percent, and even more preferably at least about 0.1 weight percent of a surfactant. For cleansing applications the personal care compositions preferably produces a Total Lather Volume of at least 300 ml, more preferably greater than 600 ml as described in the Lathering Volume Test. The personal care compositions preferably produces a Flash Lather Volume of at least 100 ml, preferably greater than 200 ml, more preferably greater than 300 ml as described in the Lathering Volume Test.

Preferable surfactants include those selected from the group consisting of anionic surfactants, nonionic surfactants, amphoteric surfactants, non-lathering surfactants, emulsifiers and mixtures thereof. Non-limiting examples of surfactants useful in the compositions of the present invention are disclosed in U.S. Pat. No. 6,280,757, to McAtee et al., issued Aug. 28, 2001.

Non-limiting examples of anionic surfactants useful in the personal care compositions of the present invention are disclosed in McCutcheon's, Detergents and Emulsifiers, North American edition (1986), published by Allured Publishing Corporation; McCutcheon's, Functional Materials, North American Edition (1992); and U.S. Pat. No. 3,929,678, to Laughlin et al., issued Dec. 30, 1975.

A wide variety of anionic surfactants are useful herein. Non-limiting examples of anionic surfactants include those selected from the group consisting of sarcosinates, sulfates, isethionates, taurates, phosphates, lactylates, glutamates, and mixtures thereof. Amongst the isethionates, the alkoyl isethionates are preferred, and amongst the sulfates, the alkyl and alkyl ether sulfates are preferred.

Other anionic materials useful herein are fatty acid soaps (i.e., alkali metal salts, e.g., sodium or potassium salts) typically having from a fatty acid having about 8 to about 24 carbon atoms, preferably from about 10 to about 20 carbon atoms. These fatty acids used in making the soaps can be obtained from natural sources such as, for instance, plant or animal-derived glycerides (e.g., palm oil, coconut oil, soybean oil, castor oil, tallow, lard, etc.) The fatty acids can also be synthetically prepared. Soaps and their preparation are described in detail in U.S. Pat. No. 4,557,853.

Other anionic materials include phosphates such as monoalkyl, dialkyl, and trialkylphosphate salts. Non-limiting examples of preferred anionic lathering surfactants useful herein include those selected from the group consisting of sodium lauryl sulfate, ammonium lauryl sulfate, ammonium laureth sulfate, sodium laureth sulfate, sodium trideceth sulfate, ammonium cetyl sulfate, sodium cetyl sulfate, ammonium cocoyl isethionate, sodium lauroyl isethionate, sodium lauroyl lactylate, triethanolamine lauroyl lactylate, sodium caproyl lactylate, sodium lauroyl sarcosinate, sodium myristoyl sarcosinate, sodium cocoyl sarcosinate, sodium lauroyl methyl taurate, sodium cocoyl methyl taurate, sodium lauroyl glutamate, sodium myristoyl glutamate, and sodium cocoyl glutamate and mixtures thereof.

Especially preferred for use herein are ammonium lauryl sulfate, ammonium laureth sulfate, sodium lauroyl sarcosinate, sodium cocoyl sarcosinate, sodium myristoyl sarcosinate, sodium lauroyl lactylate, and triethanolamine lauroyl lactylate.

Non-limiting examples of nonionic surfactants for use in the personal care compositions of the present invention are disclosed in McCutcheon's, Detergents and Emulsifiers, North American edition (1986), published by Allured Publishing Corporation; and McCutcheon's, Functional Materials, North American Edition (1992).

Nonionic surfactants useful herein include those selected from the group consisting of alkyl glucosides, alkyl polyglucosides, polyhydroxy fatty acid amides, alkoxylated fatty acid esters, sucrose esters, amine oxides, and mixtures thereof.

Non-limiting examples of preferred nonionic surfactants for use herein are those selected from the group consisting of C8-C14 glucose amides, C8-C14 alkyl polyglucosides, sucrose cocoate, sucrose laurate, lauramine oxide, cocoamine oxide and mixtures thereof.

The term “amphoteric surfactant,” as used herein, is also intended to encompass zwitterionic surfactants, which are well known to formulators skilled in the art as a subset of amphoteric surfactants.

A wide variety of amphoteric lathering surfactants can be used in the personal care compositions of the present invention. Particularly useful are those which are broadly described as derivatives of aliphatic secondary and tertiary amines, preferably wherein the nitrogen is in a cationic state, in which the aliphatic radicals can be straight or branched chain and wherein one of the radicals contains an ionizable water solubilizing group, e.g., carboxy, sulfonate, sulfate, phosphate, or phosphonate.

Non-limiting examples of amphoteric surfactants useful in the compositions of the present invention are disclosed in McCutcheon's, Detergents and Emulsifiers, North American edition (1986), published by Allured Publishing Corporation; and McCutcheon's, Functional Materials, North American Edition (1992).

Non-limiting examples zwitterionic surfactants are those selected from the group consisting of betaines, sultaines, hydroxysultaines, alkyliminoacetates, iminodialkanoates, aminoalkanoates, and mixtures thereof.

Preferred surfactants for use herein are the following, wherein the anionic surfactant is selected from the group consisting of ammonium lauroyl sarcosinate, sodium trideceth sulfate, sodium lauroyl sarcosinate, ammonium laureth sulfate, sodium laureth sulfate, ammonium lauryl sulfate, sodium lauryl sulfate, ammonium cocoyl isethionate, sodium cocoyl isethionate, sodium lauroyl isetlionate, sodium cetyl sulfate, sodium lauroyl lactylate, triethanolamine lauroyl lactylate, and mixtures thereof, wherein the non-ionic surfactant is selected from the group consisting of lauramine oxide, cocoamine oxide, decyl polyglucose, lauryl polyglucose, sucrose cocoate, C.sub.12-14 glucosamides, sucrose laurate, and mixtures thereof; and wherein the amphoteric surfactant is selected from the group consisting of disodium lauroamphodiacetate, sodium lauroamphoacetate, cetyl dimethyl betaine, cocoamidopropyl betaine, cocoamidopropyl hydroxy sultaine, and mixtures thereof.

A wide variety of non-lathering surfactants are useful herein. The personal care compositions of the present invention can comprise a sufficient amount of one or more non-lathering surfactants to emulsify the dispersed phase to yield an appropriate particle size and good application properties on wet skin.

Nonlimiting examples of these non-lathering compositions are: polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, PEG-100 stearate, polyoxyethylene 20 sorbitan trioleate (Polysorbate 85), sorbitan monolaurate, polyoxyethylene 4 lauryl ether sodium stearate, polyglyceryl-4 isostearate, hexyl laurate, steareth-20, ceteareth-20, PPG-2 methyl glucose ether distearate, ceteth-10, diethanolamine cetyl phosphate, glyceryl stearate, PEG-100 stearate, and mixtures thereof.

In addition, there are several commercial emulsifier mixtures that are useful in some embodiments of the personal care compositions according to the present invention. Examples include PROLIPID 141 (glyceryl stearate, behenyl alcohol, palmitic acid, stearic acid, lecithin, lauryl alcohol, myristyl alcohol and cetyl alcohol) and 151 (Glyceryl stearate, cetearyl alcohol, stearic acid, 1-propanamium, 3-amino-N-(2-(hydroxyethyl-)—N—N-Dimethyl,N—C(16-18) Acyl Derivatives, Chlorides) from ISP; POLAWAX NF (Emulsifying wax NF), INCROQUAT BEHENYL TMS (behentrimonium sulfate and cetearyl alcohol) from Croda; and EMULLIUM DELTA (cetyl alcohol, glyceryl stearate, peg-75 stearate, ceteth-20 and steareth-20) from Gattefosse.

The personal care compositions of the present invention, in some embodiments, may further include one or more thickening/aqueous phase stability agents. Because different stability agents thicken with different efficiencies, it is difficult to provide an accurate compositional range, however, when present, the composition preferably comprises no more than about 20 weight percent, more preferably no more than about 10 weight percent, more preferably no more than about 8 weight percent, and still more preferably no more than about 7 weight percent of the personal care composition. When present, the thickening/aqueous phase stability agent preferably comprises at least about 0.01 weight percent, more preferably at least about 0.05 weight percent, and still more preferably at least about 0.1 weight percent of the personal care composition. A better method of describing the Stability Agent is to say that it must build viscosity in the product. This can be measured using the Stability Agent Viscosity Test. Preferably, the stability agent produces a viscosity in this test of at least 1000 cps, more preferably at least 1500 cps, and still more preferably at least 2000 cps.

Nonlimiting examples of thickening agents useful herein include carboxylic acid polymers such as the carbomers (such as those commercially available under the trade name CARBOPOL®900 series from B.F. Goodrich; e.g., CARBOPOL®954). Other suitable carboxylic acid polymeric agents include copolymers of C.sub.10-30 alkyl acrylates with one or more monomers of acrylic acid, methacrylic acid, or one of their short chain (i.e., C.sub.1-4 alcohol) esters, wherein the cross linking agent is an allyl ether of sucrose or pentaerytritol. These copolymers are known as acrylates/C.sub.10-30 alkyl acrylate crosspolymers and are commercially available as CARBOPOL®1342, CARBOPOL®(1382, PEMULEN TR-1, and PEMULEN TR-2, from B.F. Goodrich.

Other nonlimiting examples of thickening agents include crosslinked polyacrylate polymers including both cationic and nonionic polymers.

Still other nonlimiting examples of thickening agents include the polyacrylamide polymers, especially nonionic polyacrylamide polymers including substituted branched or unbranched polymers. More preferred among these polyacrylamide polymers is the nonionic polymer given the CTFA designation polyacrylamide and isoparaffin and laureth-7, available under the Trade name SEPIGEL 305 from Seppic Corporation (Fairfield, N.J.). Other polyacrylamide polymers useful herein include multi-block copolymers of acrylamides and substituted acrylamides with acrylic acids and substituted acrylic acids. Commercially available examples of these multi-block copolymers include HYPAN SR150H, SS500V, SS500W, SSSA100H, from Lipo Chemicals, Inc., (Patterson, N.J.).

Another nonlimiting class of thickening agents useful herein is the polysaccharides. Nonlimiting examples of polysaccharide gelling agents include those selected from cellulose, and cellulose derivatives. Preferred among the alkyl hydroxyalkyl cellulose ethers is the material given the CTFA designation cetyl hydroxyethylcellulose, which is the ether of cetyl alcohol and hydroxyethylcellulose, sold under the trade name NATROSEL® CS PLUS from Aqualon Corporation (Wilmington, Del.). Other useful polysaccharides include scieroglucans which are a linear chain of (1-3) linked glucose units with a (1-6) linked glucose every three units, a commercially available example of which is CLEAROGEL™ CS 11 from Michel Mercier Products Inc. (Mountainside, N.J.).

Another nonlimiting class of thickening agents useful herein is the gums. Nonlimiting examples of gums useful herein include hectorite, hydrated silica, xantham gum, cellulose gums, guar gum, biosaccharide gums and mixtures thereof.

Yet another nonlimiting class of thickening agents useful herein is the modified starches. Acrylate modified starches such as WATERLOCK®from Grain Processing Corporation may be used. Hydroxypropyl starch phosphate, tradename STRUCTURE XL from National Starch is another example of a useful modified starch, and other useful examples include ARISTOFLEX HMB (Ammonium Acrylodimethyltaruate/Beheneth—25 Methacrylate Crosspolymer) from Clariant and cationic stabylens.

The personal care compositions according to the present invention may also contain organic cationic deposition polymers. Concentrations of the cationic deposition polymers preferably range from about 0.025% to about 3%, more preferably from about 0.05% to about 2%, even more preferably from about 0.1% to about 1%, by weight of the personal care composition.

Suitable cationic deposition polymers for use in the present invention contain cationic nitrogen-containing moieties such as quaternary ammonium or cationic protonated amino moieties. The cationic protonated amines can be primary, secondary, or tertiary amines (preferably secondary or tertiary), depending upon the particular species and the selected pH of the personal cleansing composition. The average molecular weight of the cationic deposition polymer is between about 5,000 to about 10 million, preferably at least about 100,000, more preferably at least about 200,000, but preferably not more than about 2 million, more preferably not more than about 1.5 million. The polymers also have a cationic charge density ranging from about 0.2 meq/gm to about 5 meq/gm, preferably at least about 0.4 meq/gm, more preferably at least about 0.6 meq/gm., at the pH of intended use of the personal cleansing composition, which pH will generally range from about pH 4 to about pH 9, preferably between about pH 5 and about pH 8.

Nonlimiting examples of cationic deposition polymers for use in the personal care compositions include polysaccharide polymers, such as cationic cellulose derivatives. Preferred cationic cellulose polymers are the salts of hydroxyethyl cellulose reacted with trimethyl ammonium substituted epoxide, referred to in the industry (CTFA) as Polyquatemium 10 which are available from Amerchol Corp. (Edison, N.J., USA) in their Polymer KG, JR and LR series of polymers with the most preferred being KG-30M.

Other suitable cationic deposition polymers include cationic guar gum derivatives, such as guar hydroxypropyltrimonium chloride, specific examples of which include the Jaguar series (preferably Jaguar C-17) commercially available from Rhodia Inc., and N-Hance polymer series commercially available from Aqualon.

Other suitable cationic deposition polymers include synthetic cationic polymers. The cationic polymers suitable for use in the personal cleansing composition herein are water soluble or dispersible, non cross linked, cationic polymers having a cationic charge density of from about 4 meq/gm to about 7 meq/gm, preferably from about 4 meq/gm to about 6 meq/gm, more preferably from about 4.2 meq/gm to about 5.5 meq/gm. The select polymers also must have an average molecular weight of from about 1,000 to about 1 million, preferably from about 10,000 to about 500,000, more preferably from about 75,000 to about 250,000.

The concentration of the cationic polymer in the personal care compositions ranges from about 0.025% to about 5%, preferably from about 0.1% to about 3%, more preferably from about 0.2% to about 1%, by weight of the composition.

A non limiting example of a commercially available synthetic cationic polymer for use in the cleansing compositions is polymethyacrylamidopropyl trimonium chloride, available under the trade name POLYCARE 133, from Rhodia, Cranberry, N.J., U.S.A.

Other non limiting examples of optional ingredients include benefit agents that are selected from the group consisting of vitamins and derivatives thereof (e.g., ascorbic acid, vitamin E, tocopheryl acetate, and the like); sunscreens; thickening agents (e.g., polyol alkoxy ester, available as CROTHIX from Croda); preservatives for maintaining the anti microbial integrity of the cleansing compositions; anti-acne medicaments (resorcinol, salicylic acid, and the like); antioxidants; skin soothing and healing agents such as aloe vera extract, allantoin and the like; chelators and sequestrants; and agents suitable for aesthetic purposes such as fragrances, essential oils, skin sensates, pigments, pearlescent agents (e.g., mica and titanium dioxide), lakes, colorings, and the like (e.g., clove oil, menthol, camphor, eucalyptus oil, and eugenol), antibacterial agents and mixtures thereof. These materials can be used at ranges sufficient to provide the required benefit, as would be obvious to one skilled in the art.

Additionally, the modified pigment particles according to present invention can be advantageously formulated into any coating or lacquer compositions including, for example, solvent-type, powder-type, emulsion-type, and aqueous-type coating compositions. Coating compositions generally contain resins, pigments, solvents, plasticizers, and other conventional additives as complicated multiple component mixtures. For example, the modified pigment particles are formulated into coating composition (i) to provide, to the coating film, color; a hiding power; physical characteristics (e.g., hardness, strength, adhesiveness); and improved weather resistance; fluorescence, phosphorescence, magnetic properties, electric conductivity, and similar inherent characteristics of the modified pigment particles, (ii) to improve the flowability of the coating composition and the workability during coating, and (iii) to prevent the generation of rust, fungal growth, and injurious organisms. For these reasons, the compatibility of pigments with resins or dispersants has been studied. Pigments have various properties, for example, from hydrophilic properties to hydrophobic properties and cause color separation and other undesirable phenomena in the resultant coating compositions. When the modified pigment particles according to the present invention are formulated into coating compositions, the unpreferable color separation does not occur because the surfaces of the modified pigments are uniformly and entirely covered with the silicone fluid layer. The resulting coating exhibits more gloss. In addition, since the surface activities of the pigments are seal coated with the silicone fluid layer, the deterioration of the coated film with the lapse of time can be effectively prevented. Furthermore, since the silicone fluid layer coated on the surface of the modified pigment particle is very thin and transparent, the basic shade of the modified pigment is not substantially changed when compared to the untreated pigment, but the intensity increases significantly and a more metallic shine is exhibited. Accordingly, color correction is not required after the modification. Typical examples of resin vehicles usable in the coating compositions according to the present invention are those conventionally used, such as, nitrocelluloses, oil modified alkyd resins, melamine resins, polyamide resins, epoxy resins, and unsaturated polyester resins.

The following examples describe certain embodiments of this invention, but the invention is not limited thereto. It should be understood that numerous changes to the disclosed embodiments could be made in accordance with the disclosure herein without departing from the spirit or scope of the invention. These examples are therefore not meant to limit the scope of the invention. Rather, the scope of the invention is to be determined only by the appended claims and their equivalents. In these examples all parts given are by weight unless otherwise indicated.

### EXAMPLE 1

### Hydrophobically Modified Silver Pearlescent Pigment Particle

A silver pearlescent pigment (mica coated with titanium dioxide, Ciba) with a particle size of less than 85 micrometers is passed through a jet mill. While passing through the jet mill, 5 percent, based on pigment weight, of cetyldimethicone (Dow Corning® 2502) is added and thoroughly mixed for twenty minutes at temperatures from 60 F to 200 F. The modified pigment particle is then sifted after treatment to remove larger particles. A hydrophobically modified silver pearlescent pigment particle is obtained.

### EXAMPLE 2

### Hydrophobically Modified Bronze Pearlescent Pigment Particle

A bronze pearlescent pigment (mica coated with iron oxide, Ciba) with a particle size of less than 85 micrometers is passed through a jet mill. While passing through the jet mill, 6 percent, based on pigment weight, of cetyldimethicone (Dow Corning® 2502) is added and thoroughly mixed for twenty minutes at temperatures from 60 F to 200 F. The modified pigment particle is then sifted after treatment to remove larger particles. A hydrophobically modified bronze pearlescent pigment particle is obtained.

### EXAMPLE 3

### Hydrophobically Modified High-Sparkling Silver Pearlescent Pigment Particle

A high-sparkling silver pearlescent pigment (mica-based bronze pearlescent pigment coated with titanium dioxide, Ciba) with a particle size of less than 150 micrometers is passed through a jet mill. While passing through the jet mill, 3 percent, based on pigment weight, of cetyidimethicone (Dow Corning® 2502) is added and thoroughly mixed for twenty minutes at temperatures from 60 F to 200 F. The modified pigment particle is then sifted after treatment to remove larger particles. A hydrophobically modified high-sparkling silver pearlescent pigment particle is obtained.

### EXAMPLE 4

### Modified Pigment Particle Incorporation into Nail Lacquers

The untreated silver pearlescent pigment and modified pearlescent pigment as described in Instant Example 1 are respectively incorporated into a nail lacquer base at 5% by weight, based on pigment weight, and stirring constantly with a propeller-type stirrer. After incorporation, both the nail lacquers are let down.

Visual assessment shows that the modified pearlescent pigment according to the instant invention exhibits significantly more gloss than the untreated sample.

### EXAMPLE 5

### Modified Pigment Application to Human Skin

A modified and untreated version of a high sparkling silver pearlescent pigment particle as described in Instant Example 3 is applied directly on the skin utilizing an eye shadow applicator. The color result appears visually significantly more intense and shimmering with the pearlescent pigment particle modified according to this invention. When rinsing with water, the untreated pearlescent pigment is washed of, while the modified pearlescent pigment stays on the skin almost unchanged, as can be visually seen.

This clearly demonstrates the unexpectedly positive results obtained by the instant invention.

### EXAMPLE 6

### Application in an Oil-in-Water Emulsion

A modified and an untreated version of a silver pearlescent pigment as described in Instant Example 1 are stirred separately into mineral oil at 10%, based on pigment weight, and this dispersion is added to an equal amount of water in a lab bottle. The bottle is closed and shaken. The untreated pearlescent pigment migrates into the water phase and the colour bleeds into the water phase, thus discolouring the aqueous phase. The modified pearlescent pigment according to the instant invention stays in the oil phase, and the colour does not bleed into the water phase, thus the water phase remains uncoloured.

This clearly demonstrates the unexpectedly positive results obtained by the instant invention.

### EXAMPLE 7

### Determination of Hydrophobicity and Skin Feel

The modified pearlescent pigment according to Instant Example 2 and the untreated version of the same bronze pearlescent pigment are added into separate small dishes and each surface is levelled with a glass plate. A drop of Castor oil is added onto the surface of each pigment powder. The castor oil droplet has a very low surface angle and is immediately soaked into the pearlescent pigment powder modified according to Instant Example 2. The pearlescent pigment powder made from the untreated pearlescent pigment exhibits a visually greater surface angle and the castor oil migrates extremely slowly into the untreated pearlescent pigment powder.

This clearly demonstrates the modified pigments according to the instant invention exhibit a great degree of hydrophobicity.

A panel of 10 individuals with expertise in skin care product evaluation is asked to apply the modified pigment according to Instant Example 3 and the untreated version of the same pearlescent pigment to the skin of the their forearms. In a side-by-side comparison, 10 out of 10 individuals found that the untreated pigment felt rough and raspy when applied to the human skin, whereas the modified pigment according to the Instant Example 3 felt smooth and silky.

This experiment clearly demonstrates the significantly better skin feel of the modified pigments according to the instant invention over untreated pigments.

### EXAMPLE 8

### Determination of Hydrophilicity

The modified pearlescent pigment according to Instant Example 2 and the untreated version of the same bronze pearlescent pigment are added into separate small dishes and each surface is levelled with a glass plate. A drop of water is added onto the surface of each pigment powder. It is observed that the water droplet is instantly absorbed by the untreated pearlescent pigment powder; while the water droplet exhibits a very high surface angle on the modified pearlescent pigment powder and does not penetrate into the pigment powder, modified according to the instant invention, at all.

This clearly demonstrates the modified pigments according to the instant invention exhibit no hydrophilicity.

### EXAMPLE 9

### Low Dusting Characteristics of Modified Pigment Particles

The modified pearlescent pigment according to Instant Example 2 and the untreated version of the same bronze pearlescent pigment (100 grams each) are dropped separately from a height of 30 cm into separate big dishes. The untreated pearlescent pigment falls in big lumps and produces great amount of dust. The modified pearlescent pigment flows better, produces significantly less dust and does not clump at all.

This clearly demonstrates the modified pigment particles according to the instant invention exhibit very low dusting when handled.

### EXAMPLE 10

### Pressed Powder Eye-Shadow

| INCI | Tradename | Supplier | Concentration |
|---|---|---|---|
| Talc | Talc Micro Ace P-2 | Presperse, Inc. | 41.50 |
| Zinc Stearate | Zinc Stearate | Witco Corp. | 5.00 |
| PTFE | Microslip 519 | Presperse, Inc. | 4.00 |
| Nylon-12 | Nylon-12 | Lipo Chemicals | 4.00 |
| Mica | Sericite PHN | Presperse, Inc. | 10.00 |
| Ultramarines | Ultramarine Blue | Sensient Technologies | 5.00 |
| Manganese Violet | Manganese Violet | Sensient Technologies | 10.00 |
| Ferric Ferrocyanide | Ferric Ferrocyanide | Sensient Technologies | 0.50 |
| Squalane | Squalane | Lipo Chemicals | 5.00 |
| Phenoxyethanol | Optiphen Plus | International Specialty | 1.00 |
| (and) Caprylyl | | Products | |
| Glycol (and) Sorbic | | | |
| Acid | | | |
| | Modified pigment | | 14.00 |
| | according to Example 3 | | |

All the ingredients are weighed in the initial phase and brought together in an osterizer or suitable grinder. When the initial phase is uniform and free of pigment specks, the binder phase is slowly added to the powder and hand mixed until the binder is completely dispersed and homogeneous. The batch is osterized again to make sure that the powder is completely wet out and the batch is uniform. The instant modified pigment is added and slowly blended to make sure it is completely uniform in the batch. Care is taken not to over grind the modified pearlescent pigment. When complete, the entire blended contents are stored in airtight containers until needed. A small amount of the batch is pressed into eye shadow pans.

### EXAMPLE 11

### O/W Foundation Makeup

| INCI | Tradename | Supplier | Concentration |
|---|---|---|---|
| Deionized Water | Deionized Water | | 63.75 |
| Cellulose Gum | CMC 7H3SF Gum | Hercules | 0.30 |
| Magnesium Aluminum | Veegum Ultra | RT Vanderbilt | 0.35 |
| Silicate | | | |
| Butylene Glycol | Butylene Glycol | Lipo Chemicals | 4.50 |
| Lecithin | Alcolec S | American Lecithin | 0.20 |
| Triethanolamine | Triethanolamine 99% | Lipo Chemicals | 1.20 |
| Titanium Dioxide | Titanium Dioxide | Kronos | 8.00 |
| Iron Oxides | Red Iron Oxide | Sun Chemical | 0.40 |
| Iron Oxides | Yellow Iron Oxide | Sun Chemical | 0.80 |
| Iron Oxides | Black Iron Oxide | Sun Chemical | 0.10 |
| Methyl Paraben | Methyl Paraben | Lipo Chemicals | 0.20 |
| Calcium Aluminum | Luxsil | Potters Industries | 2.00 |
| Borosilicate | | | |
| Isoeicosane | Permethyl 102A | Presperse Inc. | 10.00 |
| Isostearic Acid | Isostearic Acid | Lipo Chemicals | 1.00 |
| Stearic Acid | Stearic Acid | Lipo Chemicals | 2.50 |
| Glyceryl monostearate | LIPO GMS 450 | Lipo Chemicals | 1.50 |
| Tridecyl Trimellitate | Liponate TDTM | Lipo Chemicals | 1.00 |
| Glyceryl monostearate | LIPO GMS 470 | Lipo Chemicals | 1.00 |
| Propyl Paraben | Propyl Paraben | Lipo Chemicals | 0.20 |
| | Modified pigment | | 1.00 |
| | according to Instant | | |
| | Example 2 | | |

Weigh water phase ingredients in a suitable vessel and begin mixing to wet out the gums. Pregrind the color phase in an osterizer or equivalent grinder. When the gums are hydrated and the phase is uniform, add the ground color phase to the water phase and mix until all the color is dispersed. Begin to heat the water phase ingredients to 75° C. with good mixing. In a separate vessel, weight the oil phase ingredients and begin heating to 75-80° C. When both phases are uniform and at the proper temperatures, slowly add the oil phase to the water phase with continuous mixing. Mix the batch for 15 minutes and then begin cooling to 25° C. At 25° C. remove the batch and store in airtight containers until ready for filling.

### EXAMPLE 12

### Moisturizer

| Phase | INCI Name | Trade Name | Supplier | Parts |
|---|---|---|---|---|
| A | Deionized Water | DI Water | N/A | Qs to 100 |
| A | Sclerotium Gum | Tinocare GL | Ciba SC | 1.00 |
| A | | Salcare SC 96 | Ciba SC | 3.00 |
| B | PPG-3 Benzyl Ether | Crodamol STS | Croda | 5.00 |
| | Myristate | | | |
| B | Cetearyl Isononanoate | Cetiol SN | Cognis | 5.00 |
| B | | Pearlescent pigment | Ciba SC | 0.50 |
| | | according to Instant | | |
| | | Example 2 | | |
| C | Phenoxyethanol and | Optiphen Plus | ISP | 0.50 |
| | Caprylyl Glycol and | | | |
| | Sorbic Acid | | | |
| C | Fragrance | Fragrance | N/A | 0.50 |

Combine A ingredients and heat to 80° C. Add B to A

Combine C ingredients and heat to 80° C. Add C to A & B with continuous mixing for 10-15 minutes. Cool to 25° C. with good mixing. Store in airtight containers until ready for filling. Fill at room temperature.

### EXAMPLE 13

### Pearlized Lipstick

| INCI Name | Trade Name | Supplier | Parts |
|---|---|---|---|
| White Beeswax | Beeswax, white | Kahl & Co | 8.00 |
| Candelilla Wax | Candelilla Wax | Rita Corp. | 4.50 |
| Carnauba Wax | Carnauba Wax #1 | Strahl & Pitsch | 6.00 |
| Isoeicosane | Permethyl 102A | Presperse | 6.00 |
| Polyisobutene | Permethyl 104A | Presperse | 3.50 |
| Isopropyl Palmitate | Crodamol IPP | Croda | 11.00 |
| Tridecyl Trimellitate | Liponate TDTM | Lipo Chemicals | 10.00 |
| Caprylic/Capric Triglyceride | Liponate GC | Lipo Chemicals | 4.00 |
| Ethylhexyl Palmitate | Ceraphyl 368 | ISP | 8.35 |
| Propyl Paraben | Nipasol M | Clariant | 0.10 |
| Pentaerythrityl Tetra-di-t-butyl | Tinogard TT | Ciba SC | 0.05 |
| Hydroxyhydrocinnamate | | | |
| Castor Oil | Castor Oil USP | Sud Chemie | To 100 |
| Red Iron Oxide, Yellow iron | Unipure Red LC831 | Sensient | q.s. |
| Oxide, black Iron Oxide | Unipure Yellow LC181 | | |
| | Unipure Black LC989 | | |
| | Pearlescent pigment | | 10.00 |
| | according to Instant | | |
| | Example 3 | | |

Melt all solid waxes at 70-80 Degrees Celsius and add rest of ingredients under stirring once the waxes are melted. When homogeneous poor into lipstick molts and let cool down to room temperature. Remove lipsticks from molds.

### EXAMPLE 14

### Lipstick

| Phase | INCI Name | Trade Name | Supplier | Parts |
|---|---|---|---|---|
| A | Castor Oil | Lipovol CO | Lipo | 33.25 |
| A | Triethylhexanoin | Schercemol GTO | Scher | 7.50 |
| A | Triisostearyl Trilinoleate | Schercemol TIST | Scher | 15.00 |
| A | Triisostearyl Citrate | Schercemol TISC | Scher | 17.50 |
| A | Euphorbia Cerifera | Refined Candelilla Wax Prills | Ross Waxes | 7.00 |
| | (Candelilla) Wax | | | |
| A | Copernicia Cerifera | Yellow Carnuba Wax Flakes | Ross Waxes | 1.80 |
| | (Carnauba) Wax | | | |
| A | Ozokerite | White Ozokerite Wax 77W | Ross Waxes | 1.80 |
| A | Microcyrstalline Wax | Microcrystalline Wax 1275W | Ross Waxes | 3.50 |
| A | Hydroxylated Lanolin | Ritahydrox | Rita | 1.00 |
| A | Methylparaben | Nipagin M | Clariant | 0.20 |
| A | Propylparaben | Nipasol M | Clariant | 0.10 |
| B | Iron Oxides | Modified Red Pigment | Ciba Specialty | 5.70 |
| | | (according to instant invention) | Chemicals | |
| B | Iron Oxides | Modified Yellow Pigment | Ciba Specialty | 1.10 |
| | | (according to instant invention) | Chemicals | |
| B | Ultramarines | Modified Blue Pigment | Ciba Specialty | 0.20 |
| | | (according to instant invention) | Chemicals | |
| B | Mica and titanium dioxide | Modified pigment according to | Ciba Specialty | 4.35 |
| | | Example 3 | Chemicals | |
| | | | Total | 100.00 |

### Procedure:

Phase A is combined, heated between 90-105° C., and mixed until uniform. Phase B is then added with stirring until homogenous. The temperature is maintained above 70° C. as the lipstick is poured into the mold.

### EXAMPLE 15

### Medium Protection Sunscreen

| Phase | INCI Name | Trade name | Supplier | Parts |
|---|---|---|---|---|
| A | Deionized Water | DI Water | N/A | 84.86 |
| A | Propylene Glycol (and) | Germaben II | ISP | 1.00 |
| | Diazolidinyl Urea (and) | | | |
| | Methylparaben (and) | | | |
| | Propylparaben | | | |
| A | Aloe Barbadensis Leaf | Aloe Gel 1:1 Natural | Tri-K Industries | 1.00 |
| | Juice | | | |
| A | Propylene Glycol | Propylene Glycol | Dow Chemical | 2.50 |
| A | Butylene Glycol (and) | Actiphyte of Black | Active Organics | 0.04 |
| | Water (and) Juglans Nigra | Walnut Hull | | |
| | (Black Walnut) Shell Extract | | | |
| A | Ethyhexyl Salicylate | Escalol 587 | ISP | 5.00 |
| A | Ethylhexyl | Escalol 557 | ISP | 3.00 |
| | Methoxycinnamate | | | |
| B | Sodium Acrylates | Ciba ® SALCARE ® SC91 | Ciba Specialty | 2.00 |
| | Copolymer (and) | | Chemicals | |
| | Paraffinum Liquidum (and) | | | |
| | PPG-1 Trideceth-6 | | | |
| C | Iron Oxides | Modified Red Pigment | Ciba Specialty | 0.20 |
| | | (according to instant | Chemicals | |
| | | invention) | | |
| C | Mica and iron oxide | Modified pigment | Ciba Specialty | 0.10 |
| | | according to Example 2 | Chemicals | |
| D | Fragrance | Flowers in the mist | Belle Aire | 0.30 |
| | | | Fragrances | |
| | | | Total | 100.00 |

### Procedures:

In an appropriate vessel add Part A and start moderate agitation.

Add part B and mix until uniform.

Add Part C, than part D and mix until well blended.

### EXAMPLE 16

### Talc Free Loose Face Powder

| Phase | INCI Name | Trade Name | Supplier | Parts |
|---|---|---|---|---|
| A | Mica | Sericite PHN | Presperse | 81.45 |
| A | Polymethyl Methacrylate | Ganzpearl GM-0600W | Presperse | 5.00 |
| A | Synthetic Wax and Corn | Microease 110XF | Presperse | 2.00 |
| | Gluten Protein | | | |
| A | Titanium Dioxide | Titanium Dioxide 3228 | Whittaker, | 5.00 |
| | | | Clark & | |
| | | | Daniels | |
| A | Methylparaben | Nipagin M | Clariant | 0.20 |
| A | Propylparaben | Nipasol M | Clariant | 0.10 |
| A | Imidazolidinyl Urea | Germall 115 | ISP | 0.25 |
| B | iron oxide | Modified Red Pigment | Ciba Specialty | 1.00 |
| | | (according to instant | Chemicals | |
| | | invention) | | |
| B | Iron Oxide | Modified Yellow Pigment | Ciba Specialty | 5.00 |
| | | (according to instant | Chemicals | |
| | | invention) | | |
| | | | Total | 100.00 |

### Procedure:

Mill together A until fully dispersed. Add B to A and blend until uniform.

### EXAMPLE 17

### Oil in Water Facial Foundation

| Phase | INCI Name | Trade Name | Supplier | Parts |
|---|---|---|---|---|
| A | Deionized water | DI Water | N/A | 53.94 |
| A | 10% KOH solution | 10% KOH solution | N/A | 1.30 |
| A | PEG-12 Dimethicone | DC 193 Surfactant | Dow Corning | 0.10 |
| A | Talc | Talc | Whittaker, | 0.72 |
| | | | Clark & | |
| | | | Daniels | |
| B | 1,3-Butylene Glycol | Jeechem BUGL | Jeen Int. | 4.00 |
| B | Magnesium Aluminum Silicate | Veegum Granules | R. T. | 1.00 |
| | | | Vanderbilt | |
| C | 1,3-Butylene Glycol | Jeechem BUGL | Jeen Int. | 2.00 |
| C | Cellulose Gum | CMC 7MF | Hercules | 0.12 |
| C | Methylparaben | Nipagin M | Clariant | 0.02 |
| D | Di-PPG-3 Myristyl Ether Adipate | Cromollient DP3-A | Croda | 14.00 |
| D | Diethyl Hexyl Maleate | Pelemol DOM | Phoenix | 4.00 |
| D | Steareth-10 | Lipocol S-10 | Lipo | 2.00 |
| D | Steareth-2 | Lipocol S-2 | Lipo | 0.50 |
| D | Cetyl Alcohol | Crodacol C-95 NF | Croda | 0.62 |
| D | Dicetyl Phosphate and | Crodafos CES | Croda | 4.00 |
| | Ceteth-10 Phosphate and | | | |
| | Ceteryl Alcohol | | | |
| D | Propyl Paraben | Nipasol M | Clariant | 0.10 |
| E | Titanium Dioxide | Modified TiO2 (according to | Ciba Specialty | 7.50 |
| | | instant invention) | Chemicals | |
| E | Mica and titanium dioxide | Modified pigment according | Ciba Specialty | 2.50 |
| | | to Example 1 | Chemicals | |
| E | Iron Oxide | Modified Red Pigment | Ciba Specialty | 1.20 |
| | | (according to instant | Chemicals | |
| | | invention) | | |
| E | Ultramarine | Blue Pigment | Ciba Specialty | 0.20 |
| | | | Chemicals | |
| F | DMDM Hydantoin | Mackstat DM | McIntyre | 0.18 |
| | | | Group | |
| | | | Total | 100.00 |

### Procedure:

Combine ingredients in phase A using a homogenizer and begin heating to 80° C. Add phase B and C and homogenize for 1 hour. In a separate beaker combine ingredients in phase D, heat to 80° C. and mix until uniform. After all ingredients in phase D have become uniform slowly add to the main phase while continuing to homogenize. Upon complete addition of phase D, homogenize for 15 min at 80° C. then begin cooling the mixture. At 60° C. switch to paddle mixing using moderate agitation. Phase E is added and mixed until homogenous mixture obtained. At 50° C. phase F is added. The mixture is cooled until it reaches room temperature.

### EXAMPLE 18

### Press Powder Eye Shadow

### Red

| INCI Name | Trade Name | Supplier | Parts |
|---|---|---|---|
| Mica | Sericite PHN | Presperse | 75.60 |
| Zinc Stearate | Zinc Stearate | Witco | 5.00 |
| Titanium Dioxide | Modified TiO2 (according to | Ciba Specialty | 6.00 |
| | instant invention) | Chemicals | |
| Iron Oxide | Modified Red Pigment | Ciba Specialty | 2.00 |
| | (according to instant | Chemicals | |
| | invention) | | |
| mica and titanium dioxide | Modified pigment according | Ciba Specialty | 0.60 |
| | to Example 3 | Chemicals | |
| Methylparaben | Nipagin M | Clariant | 0.20 |
| Propylparaben | Nipasol M | Clariant | 0.10 |
| Calcium Aluminum Borosilicate | Luxsil | Presperse | 5.00 |
| PEG-4 Diheptanoate | Liponate 2-DH | Lipo | 5.50 |
| | | Total | 100.0 |

### Procedure:

Combine ingredients and mix well. Heat to 100° C. and press at 2000 psi.

### EXAMPLE 19

### Nail Enamel

### Purple

| Phase | INCI Name | Trade name | Supplier | Parts |
|---|---|---|---|---|
| A | Butyl Acetate and Toluene | Suspending Lacquer | Engelhard | 86.00 |
| | and Nitrocellulose and | SLF-2 | | |
| | Tosylamide/Formadlehyde | | | |
| | Resin and Isopropyl Alcohol | | | |
| | and Dibutyl Phthalate and | | | |
| | Ethyl acetate and Camphor | | | |
| | and n-Butyl Alcohol and | | | |
| | Silica and Quaterinium-18 | | | |
| | Hectorite | | | |
| A | Butyl Acetate (and) Bismuth | Biju Ultra UXD | Engelhard | 2.25 |
| | Oxychloride (and | | | |
| | Nitrocellulose (and) | | | |
| | Isopropyl Alcohol (and) | | | |
| | Stearylalkonium Hectorite | | | |
| A | Mica (and) Titanium Dioxide | Flamenco Ultra | Engelhard | 1.00 |
| | | Sparkle 4500 | | |
| A | Colorant | Modified Red | Ciba Specialty | 1.25 |
| | | Pigment (according | Chemicals | |
| | | to instant invention) | | |
| A | Colorant | Modified Blue | Ciba Specialty | 1.25 |
| | | Pigment (according | Chemicals | |
| | | to instant invention) | | |
| A | Dimethicone | Dow Corning 200 | Dow Corning | 1.00 |
| A | Tosylamide/Epoxy Resin | Lustrabrite S-70 | Telechemische | 4.00 |
| B | Butyl Acetate | Butyl Acetate | Dow Chemical | 1.17 |
| B | Ethyl Acetate | Ethyl Acetate | Dow Chemical | 0.42 |
| B | Toluene | Toluene | Shell | 1.66 |
| | | | Total | 100.00 |

### Procedure:

Combine phase A and mix until uniform. Combine phase B in a separate vessel and mix until uniform. Add phase B to phase A with stirring until uniform.

### EXAMPLE 20

### Hydrophobically Modified Silver Pearlescent Pigment Particle

A silver pearlescent pigment (mica coated with titanium dioxide, Ciba) with a particle size of less than 85 micrometers is passed through a jet mill. While passing through the jet mill, 5 percent, based on pigment weight, of a cetyldimethicone/dimethylpolysiloxane (1:1 weight ratio) is added and thoroughly mixed for twenty minutes at temperatures from 72 F to 180 F. The modified pigment particle is then sifted after treatment to remove larger particles. A hydrophobically modified silver pearlescent pigment particle is obtained.

What is claimed is:

1. A method for the hydrophobic treatment of pigment particles comprising contacting said pigment particles with an effective amount of one or more silicone compounds having formula (1), (2), and (3): 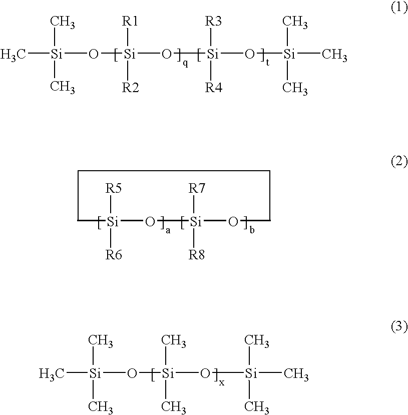
wherein R1 is C1 to C22 branched or straight chain alkyl or aryl of six to ten carbon atoms; R2 is C1 to C22 branched or straight chain alkyl or aryl of six to ten carbon atoms; R3 is C1 to C22 branched or straight chain alkyl or aryl of six to ten carbon atoms; R4 is C11 to C22 branched or straight chain alkyl or aryl of six to ten carbon atoms; q is from 1 to about 2000; t is from 1 to about 2000; the sum of q+t is from 2 to about 2000;
R5 is C1 to C22 branched or straight chain alkyl or aryl of six to ten carbon atoms; R6 is C1 to C22 branched or straight chain alkyl or aryl of six to ten carbon atoms; R7 is C1 to C22 branched or straight chain alkyl or aryl of six to ten carbon atoms; R8 is C1 to C22 branched or straight chain alkyl or aryl of six to ten carbon atoms; a is from 1 to about 100; b is from 1 to about 100; the sum of a+b is from 2 to about 100;
x is an integer from 1 to about 2000;
with the proviso that formula (1), (2), and (3) are essentially free of silicon-hydrogen bonds and fluorine and with the further proviso that one of the silicone compounds is of formula (1).

2. A method according to claim 1 wherein the silicone compound of formula (1) is defined as:
R1 is C1 to C10 branched or straight chain alkyl or aryl of six to ten carbon atoms; R2 is C1 to C10 branched or straight chain alkyl or aryl of six to ten carbon atoms; R3 is C1 to C10 branched or straight chain alkyl or aryl of six to ten carbon atoms; R4 is C12 to C18 branched or straight chain alkyl or aryl of six to ten carbon atoms; q is from 1 to about 1000; t is from 1 to about 1000; the sum of q+t is from 2 to about 1000.

3. A method according to claim 2 the silicone compound of formula (1) is defined as:
R1 is C1 to C6 branched or straight chain alkyl; R2 is C1 to C6 branched or straight chain alkyl;
R3 is C1 to C16 branched or straight chain alkyl; R4 is C12 to C18 branched or straight chain alkyl; q is from 1 to about 100; t is from 1 to about 100; the sum of q+t is from 2 to about 100.

4. A method according to claim 3 wherein the silicone compound of formula (1) is cetyldimethicone.

5. A method according to claim 1 wherein the silicone compound of formula (1) is cetyldimethicone and a silicone compound of formula (2) wherein the silicone compound of formula (2) is defined as:
R5 is methyl; R6 is methyl; R7 is methyl; R8 is C1 to C8 straight chain alkyl; a is from 1 to about 9; b is from 1 to about 9; the sum of a+b is from 3 to about 9.

6. A method according to claim 5 wherein the silicone compound of formula (1) is cetyldimethicone and a silicone compound of formula (2) wherein the silicone compound of formula (2) is selected from the group consisting of cyclotetradimethylsiloxane, cyclopentadimethylsiloxane, cyclohexadimethylsiloxane, cyclocopolymer of dimethylsiloxane/methyloctylsiloxane, hexylheptamethyltrisiloxane, and octylheptamethyltrisiloxane.

7. A method according to claim 1 wherein the silicone compound of formula (1) is cetyldimethicone and a silicone compound having formula (3) wherein the silicone compound of formula (3) is defined as:
x is an integer from 1 to about 50.

8. A method according to claim 7 wherein the silicone compound of formula (1) is cetyldimethicone and a silicone compound having formula (3) wherein the silicone compound of formula (3) is dimethylpolysiloxane.

9. A method according to claim 1 wherein the pigment particles are selected from the group consisting of inorganic pigments, metal oxides and hydroxides, mica, organic pigments, pearlescent pigments, mineral silicates, porous materials, carbons, and interference pigments.

10. A method according to claim 9 wherein the pigment particles are pearlescent pigments or interference pigments.

11. A method according to claim 1 wherein the effective amount of one or more silicone compounds, based on the total weight of treated pigment particles, used is from about 0.1 percent to about 90 percent.

12. A method according to claim 11 wherein the effective amount of one or more silicone compounds, based on the total weight of treated pigment particles, used is from about 1 percent to about 15 percent.

13. A method according to claim 1 wherein pigment particles have an average particle size from about 0.01 micrometers to about 500 micrometers.

14. A method according to claim 13 wherein pigment particles have an average particle size from about 5 micrometers to about 150 micrometers.

15. A method according to claim 1 wherein the weight ratio of formula (1) to formula (2) and/or formula 3 is from about 1000:1 to about 1:1000.

16. A method according to claim 15 wherein the weight ratio of formula (1) to formula (2) and/or formula 3 is from about 10:1 to about 1:10.

17. A coated pigment particle comprising a pigment particle and the coating on said pigment particle wherein said coating comprises an effective amount one or more silicone compounds having formula (1), (2), and (3) 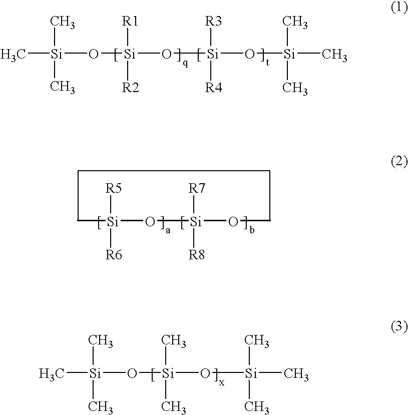
wherein R1 is C1 to C22 branched or straight chain alkyl or aryl of six to ten carbon atoms; R2 is C1 to C22 branched or straight chain alkyl or aryl of six to ten carbon atoms; R3 is C1 to C22 branched or straight chain alkyl or aryl of six to ten carbon atoms; R4 is C11 to C22 branched or straight chain alkyl or aryl of six to ten carbon atoms; q is from 1 to about 2000; t is from 1 to about 2000; the sum of q+t is from 2 to about 2000;
R5 is C1 to C22 branched or straight chain alkyl or aryl of six to ten carbon atoms; R6 is C1 to C22 branched or straight chain alkyl or aryl of six to ten carbon atoms; R7 is C1 to C22 branched or straight chain alkyl or aryl of six to ten carbon atoms; R8 is C1 to C22 branched or straight chain alkyl or aryl of six to ten carbon atoms; a is from 1 to about 100; b is from 1 to about 100; the sum of a+b is from 2 to about 100;
x is an integer from 1 to about 2000;
with the proviso that formula (1), (2), and (3) are essentially free of silicon-hydrogen bonds and fluorine and with the further proviso that one of the silicone compounds is of formula (1).

18. A personal care composition comprising coated pigment particles according to claim 17.

19. A personal care composition according to claim 18 wherein coated pigment particles comprise from about 0.01 percent to about 100 percent based on weight of the personal care composition.

20. A personal care composition according to claim 19 wherein said coated pigment particles comprise from about 0.01 percent to about 50 percent based on weight of the personal care composition.

21. A personal care composition according to claim 20 wherein said coated pigment particles comprise from about 0.2 percent to about 5 percent based on weight of the personal care composition.

22. A personal care composition according to claim 18 wherein the personal care composition is in the form of a shaving preparation, a skin-care preparation, a cosmetic personal care preparation, a light-protective preparation, a manicure preparation, a deodorant, antiperspirant, or a personal cleaning product.

23. A personal care composition according to claim 18 which is formulated as a water-in-oil or oil-in-water emulsion, as an alcoholic or alcohol-containing formulation, as a vesicular dispersion of an ionic or non-ionic amphiphilic lipid, as a gel, or a solid stick.

24. A personal care composition according to claim 18, which further comprises at least one further constituent selected from the group consisting of sequestering agents, non-encapsulated colorings, perfumes, thickening or solidifying (consistency regulator) agents, emollients, UV absorbers, surfactants, bleaching agents, skin-protective agents, antioxidants and preservatives.

25. A personal care composition according to claim 18, wherein the personal care composition comprises more than one coated pigment particle.
